(19) United States
(12) Patent Application Publication
(10) Pub. No.: US 2007/0249986 A1
(43) Pub. Date: Oct. 25, 2007

(54) Arteriovenous access for hemodialysis employing a vascular balloon catheter and an improved hybrid endovascular technique

(76) Inventor: Douglas R. Smego, Greenwich, CO (US)

Correspondence Address: David Prashker, Esq., 8 Chateau Heights, Magnolia, MA, 01930-5289, US

(21) Appl. No.: 11/707,218

(22) Filed: Feb. 15, 2007

Related U.S. Application Data
(63) Continuation-in-part of application No. 11/074,384, filed on Mar. 7, 2005.
(60) Provisional application No. 60/553,007, filed on Mar. 15, 2004.

(51) Int. Cl. A61M 5/00

(52) U.S. Cl. 604/8

(57) ABSTRACT

The present invention provides a kit apparatus and a methodology to prevent the primary causes of arteriovenous graft thrombosis; and provides a durable vascular access for successful long term use in hemodialysis. The invention employs a patient-customized prosthetic endograft as an subcutaneously implanted vascular access; and utilizes a surgical method for endovascular insertion of the prosthetic endograft into a pre-chosen vein, which does not require a distal anastomosis, and thus allows the distal outflow end of the implanted vascular access to remain unattached and freely floating at a precisely located anatomic position within the internal lumen the pre-chosen vein.

### CROSS-REFERENCE

This application is a Continuation-In-Part of U.S. patent application Ser. No. 11/074,384 filed Mar. 7, 2005. The filing date and priority benefit of this earlier filing is expressly claimed pursuant to 35 U.S.C. 120.

### FIELD OF THE INVENTION

This invention relates generally to the making of a permanent anatomic connection to access the vascular blood system in-vivo; and is directed specifically to a hybrid endovascular technique for creating an arteriovenous access suitable for hemodialysis in humans.

### BACKGROUND OF THE INVENTION

Renal disease continues to be an important cause of mortality and morbidity in the United States and throughout the world. Renal disease may be acute or chronic. Acute renal failure is a worsening of renal function over hours to days, resulting in the retention of nitrogenous wastes (such as urea nitrogen) and creatinine in the blood. In comparison, chronic renal failure results from a loss of renal function over months to years. It is presently estimated that between 4-5% of the entire American population have some form of kidney disease; and that over four hundred thousand persons in America reach that life threatening medical condition or clinical stage known as End Stage Renal Disease (or “ESRD”) which signifies the complete lack of life preserving renal function in that person.

Based upon 2002 data from the CMS, the National Kidney Foundation and the End Stage Renal Disease Network, there are approximately 406,000 patients with end stage renal disease in the United States. Yet in 1990, the same sources utilizing the same definitions and processes estimated just over 200,000 patients with end stage renal disease. Thus, the rate is more than twice the incidence reported about ten years previously, and reveals that more than ninety thousand new patients are diagnosed with ESRD each year.

Unquestionably, there has been a constant increase in the number of patients with renal disease of some variety, now estimated at 4.45% of the entire population. The largest percentages increases have been seen in the group of patients requiring treatment for end stage renal disease; and it is the elderly population which has seen the largest increases in renal disease and in end stage renal disease particularly.

### A. End Stage Renal Disease

Persons suffering from End Stage Renal Disease (“ESRD”) constitute a particular class of medical patients which require renal replacement therapy, either in the form of blood dialysis or kidney transplantation, in order to survive. A healthy kidney functions to remove toxic wastes and excess water from the blood. However, with End Stage Renal Disease (“ESRD”), there is chronic kidney failure; and the kidneys progressively fail and stop performing their essential functions over an extended period of time. If and when the kidneys progressively continue to fail in this manner, the patient afflicted with ESRD will die within a short period of time (usually hours or days) unless (i) that patient receives blood dialysis treatment quickly, a process which must then be continued and repeatedly performed at regular time intervals for the rest of that patient's life; or (ii) the patient undergoes transplantation therapy and receives a healthy and biocompatible, normal kidney from a donor. Unfortunately, because relatively few kidneys are presently available for transplantation purposes, the overwhelming majority of patients suffering from ESRD must receive regular blood dialysis treatments for the remainder of their lives.

It will be recognized also that the present rate of human ESRD is more than twice the incidence rate reported ten years ago, with more than ninety thousand new ESRD patients being diagnosed each year. The majority of these patients range from 45-64 years of age (40.9% of the class) or from 65-74 years of age (19.8% of the class). ESRD affects males (55% of the class) more than females (45% of the class); and afflicts Caucasians patents (60% of the class) more than twice as often as black/African-American patients (32% of the class). Lastly, the price for medically treating ESRD continues to rise; for example, the cost to the Federal government for the medical management of ESRD is currently 17.9 billion dollars annually.

### B. Hemodialysis

Currently, hemodialysis is the primary modality of therapy for patients with ESRD. A hemodialysis machine pumps blood from the patient, through a dialyzer, and then back into the patient. Hemodialysis therapy is thus an extracorporeal (i.e., outside the body) process which removes toxins and water from a patient's blood; and requires a constant flow of blood along one side of a semipermeable membrane with a cleansing solution, or dialysate, on the other. Diffusion and convection allow the dialysate to remove unwanted substances from the blood while adding back needed components. In this manner, the dialyzer removes the toxins and water from the blood by a membrane diffusion principle.

Hemodialysis is most often performed as an out patient procedure in approximately 3,600 approved centers in the U.S. In comparison, home dialysis is an option that is becoming ever less popular because of the need for a trained helper, large-sized dialysis equipment, and the very high costs. Typically, a patient with ESRD disease requires hemodialysis three times per week. Each session usually lasts for 3-6 hours depending on patient size, type of dialyzer employed and other medical factors.

### C. The Need for a Vascular Access

Removing blood from the body in order to filter the blood in the dialysis process requires a vascular access to the patient's blood system. A vascular access can be obtained in the short term via the use of percutaneous implanted catheters; but such short-term apparatus and methods ultimately must be replaced by long term procedures—which typically include surgically modifying the patient's own blood vessels to create an arteriovenous (“A-V”) fistula or surgically implanting a pre-formed prosthetic graft into the individual's blood vessels. In these long-term techniques, the vascular access site (such as the A-V fistula or prosthetic graft) lies entirely beneath the skin; and the skin and the internalized vascular access site must thus be punctured externally from outside the body using a syringe needle and blood tubing which is joined to the dialysis machine.

To be medically useful, the chosen mode of vascular access must remain patent (i.e., unblocked) and remain free from medical complications in order to enable dialysis to take place. The vascular access must also allow blood to flow to and return from the dialysis machine at a sufficiently high rate to permit dialysis to take place efficiently; and, desirably, it should allow the patient to carry on at least the semblance of a normal life.

However, the vascular access is widely called the “Achilles heel of dialysis” because of the markedly high morbidity and mortality among dialysis patients associated with complications of vascular access. Vascular access complications are believed to be the single greatest cause of morbidity; and, moreover, are believed to account for approximately one-fourth of all admissions and hospitalization days in the ESRD population.

For example, of those patients afflicted with end stage renal disease (about 293,000 persons) receiving hemodialysis at any given time, only 39% of them (about 113,000 persons) are believed to have a working vascular access graft suitable for maintenance dialysis. The remaining 180,000 patients typically require the placement of temporary percutaneous vascular access catheters as they are awaiting placement, or revision, and/or maturation of a permanent vascular access graft. In addition, it is estimated that a minimum of 2500 new patient vascular access grafts are placed each year, with an optimal longevity of 3 years time before revision is necessary. Thus, a cycle of vascular access graft placement, a period of successful utilization, followed by intercurrent thrombosis, graft revision, and ultimate failure and replacement occurs during the remainder of the entire life of these patients.

Moreover, each time a new vascular access graft is placed or replaced, the prosthetic materials cost approximately one thousand (US) dollars. This cost is, of course, added to the hospitalization, operating room, drug, and related physician costs; as well as to the costs of instituting and maintaining the required temporary vascular access prior to and immediately following the permanent vascular access graft placement.

Consequently, by virtue of the recurring pattern of pathophysiology for the A-V access in humans, multiple revisions and replacement of the access itself is the rule in vascular access surgery. This combination of natural history failures, co-morbidity, and complications of therapy today results in approximately 67,000 deaths attributed to ESRD in the U.S. alone.

The medical and scientific literature evidences the severity of the problem. Merely illustrative of such medical and scientific printed publications are the following: Sidawy et al., “Seminars in Vascular Surgery”, AV Hemodialysis Access and its Management, Vol 17, No. 1, March 2004; Gibson et al, “Vascular access survival and incidence of revisions: A comparison of prosthetic grafts, simple autogenous fistulas, and venous transposition fistulas from the U.S. Renal Data System Dialysis Morbidity and Mortality Study”, J Vasc Surg 34:694-700 (2001); The Vascular Access Work Group, “NfK-DOQI clinical practice guidelines for vascular access”, Am I Kidney Dis 37(suppl. 1):s137-sl81 (2001); Puskas J. D. and J. P. Gertler, “Internal jugular to axillaiy vein bypass for subclavian vein thrombosis in the setting of brachial a-v fistula”, J Vasc Surg 19:939-942 (1994); Fulks et al., “Jugular-axillary vein bypass for salvage of a-v access”, J Vasc Surg 9:169-171 (1980); Collins et al., “United States Renal Data System assessment of the impact of the National Kidney Foundation-Dialysis Outcomes Quality Initiative guidelines”, Am J Kidney Dis 39:784-795 (2002); Kalrnan et al., “A practical approach to vascular access for hemodialysis and predictors of success”, J Vasc Surg 30:727-733 (2004); Palder et al., “Vascular access for hemodialysis: Patency rates and results of revision”, Ann Surg 202:235-239 (1985); Scher et al., “Alternative graft materials for hemodialysis access”, Sem Vasc Surg 17(1):19-24 (2004); and Schuman et al., “Reinforced versus nonreinforced ptfe grafts for hemodialysis access”, Am J Surg 173:407-410 (1997).

### D. The Conventionally Known Means for Providing a Vascular Access

The need for vascular access in patients with renal failure can be either temporary or permanent. Devices and methods are available today to establish temporary vascular access for time periods ranging from several hours to several weeks. In comparison, permanent access methods and devices allow vascular access to a patient's blood system which typically last for months to years in duration.

In good medical practice, a temporary vascular access is typically used to treat patients with acute renal failure; patients in chronic renal failure without an available mode of permanent access; peritoneal dialysis patients or transplant patients needing temporary hemodialysis; and patients requiring plasmapheresis or hemoperfusion. In contrast, permanent vascular access devices and methods are the requisite rule for patients suffering from end stage renal disease.

A listing of the historically known, major kinds of vascular access is given below:

| Device & | | Year Of First |
|---|---|---|
| Technique | Type | Introduction |
| 1. Scribner shunt | Temporary Access | 1959/1960 |
| 2. Percutaneous catheter | Temporary Access | 1983 |
| assembly | | |
| 3. A-V (arteriovenous) | Permanent Access | 1966 |
| fistula | | |
| 4. Polytetrafluoroethylene | Permanent Access | 1977 |
| (PTFE) graft | | |

The Scribner Shunt:

The Scribner shunt was the earliest developed breakthrough percutaneous device which allowed patients afflicted with chronic kidney disease to have a temporary vascular access and the ability to be treated with the relatively primitive hemodialysis machines already-existing at that time. The device is an externally located arteriovenous shunt, developed in 1960 by Quinton and Shribner; and consists of two hard plastic cylinders or vessel tips. One vessel tip is implanted into an extremity artery and the other into a nearby vein; and the opposite vessel tip ends are connected to pieces of silicone elastomer tubing. After implantation, the two silicone tubes are connected with each other to establish the external shunt [see for example: E. Larson, L. Lindbloom and K. B. Davis, Development of the Clinical Nephrology Practitioner, Mosby, St. Louis, 1982; J. T. Daugirdas and T. S. Ing, Handbook of Dialysis, 2^{nd} Ed., Little, Brown and Co., 1994].

The Scribner shunt suffered from major infection and clotting problems; and also required extensive post-operative and long-term care of the shunt. For these reasons, the Scribner shunt is today largely obsolete and is no longer used for hemodialysis.

### The Percutaneous Catheter Assembly

The second temporary method of vascular access is a percutaneous venous cannula assembly which is inserted into a major vein—such as the femoral, subclavian or jugular vein. These catheter assemblies are percutaneous, with one end lying external to the body and the other end typically dwelling internally within either the superior vena cava or the right atrium of the heart. The external portion of these catheter assemblies has connectors permitting attachment of blood sets leading to and from a hemodialysis machine.

Typically, a percutaneous catheter assembly is a venous cannula having a catheter element and a connector portion comprising an extracorporeal connector element. In usual practice, the assembly's extracorporeal connector element is disposed against the chest of the patient; and the distal end of the catheter element is passed into a pre-chosen internal vein; and then is passed down through the vein into the patient's superior vena cava. More particularly, the distal end of the catheter element is usually positioned within the patient's superior vena cava such that the mouth of the suction line, as well as the mouth of the return line, are both located between the patient's right atrium and the patient's left subclavian vein and right subclavian vein. The percutaneous venous cannula assembly is then left in this position relative to the body, ready and waiting to be used during an active dialysis session.

Manner of Use

When hemodialysis is to be performed on the patient, the assembly's extracorporeal connector element is appropriately connected to a dialysis machine,—i.e., the suction line is connected to the input port (the suction port) of the dialysis machine; and the return line is connected to the output port (the return port) of the dialysis machine. The dialysis machine is then activated—i.e., the dialysis machine's blood pump is turned on and the flow rate set. The dialysis machine will withdraw relatively “dirty” blood from the patient through the suction line and return relatively “clean” blood to the patient through the return line. In practice, it has generally been found desirable to separate the assembly's two mouths by a distance of about 2, inches or so in order to avoid such undesired blood recirculation.

Perspective Changes Over Time

Percutaneous catheter assemblies have been used in hemodialysis since the early 1960's but for many years have been considered to be only a “temporary” form of vascular access because of their concomitant major infection and stenosis problems. However, because they can be easily and quickly inserted, they were used when emergency vascular access was needed to permit hemodialysis. Nevertheless, for many years, the risk of potentially life-threatening infection complications was considered to be so great that the percutaneous catheter assemblies were withdrawn after each dialysis session and re-inserted when necessary to minimize the risk of infection.

Yet, despite this history, two important developments occurred in the 1980's that have led some nephrologists to consider using percutaneous catheter assemblies as a “permanent” form of vascular access. The first and most important of these developments was a 1983 paper reporting the insertion of percutaneous catheter assemblies into the jugular vein rather than the subclavian vein. Jugular vein insertion essentially eliminated the problem of subclavian vein stenosis associated with up to 50% of subclavian vein catheter insertions. Note that subclavian vein stenosis not only blocks blood flow, making it impossible to conduct hemodialysis; but also, catastrophically, can destroy all potential vascular access sites in one or both arms.

The second major development was the attachment of a Dacron “cuff” to the assembly's catheter element, near the proximal end, under the skin, about an inch from the incision site where the assembly exits the body. This cuff permits tissue in-growth to occur, which fastens the catheter element to the tissue and thereby reduces movement of the percutaneous catheter assembly at the incision site as well as in the blood vessel. In addition, such tissue in-growth is believed by many medical practitioners to retard bacterial travel along the outer surface of the percutaneous catheter assembly, although it does not prevent it entirely. Yet, while numerous published reports suggest that the cuff has reduced the infection rate, clinical infections remain a major problem even with the use of cuffed percutaneous catheter assemblies.

Nevertheless, because of these developments, a series of papers published in the 1990's reported positively on the long term survival of percutaneous catheter assemblies—thereby permitting and openly encouraging their use as a “permanent” form of vascular access. In addition, a wide range and variety of catheter apparatus improvements and catheterization method innovations have been generated which intend that venous cannula assemblies be employed as “permanent” means of vascular access. Merely exemplifying some of the most recent of these apparatus improvements and method of use innovations are the following: U.S. Pat. No. 6,758,841 entitled “Percutaneous Access”; U.S. Pat. No. 6,758,836 entitled “Split Tip Dialysis Catheter”; U.S. Pat. No. 6,685,664 entitled “Method And Apparatus For Ultrafiltration Utilizing A Long Peripheral Access Venous Cannula For Blood Withdrawl”; and U.S. Pat. No. 6,620,118 entitled “Apparatus And Method For The Dialysis Of Blood”. Each of these issued patents as well as the publications cited internally within them are expressly incorporated by reference herein.

### The A-V (Arteriovenous) Fistula

A major method of permanent vascular access currently in use is the A-V (arteriovenous) fistula. By definition, an A-V fistula is a naturally occurring linkage or a surgical construct connecting a major artery to a major vein subcutaneously. For hemodialysis purposes, an anatomically-sited and purposefully created surgical construction is the practical reality.

A primary arteriovenous fistula is a preferred and cost-effective long-term access for hemodialysis patients. Because an A-V fistula is an artificial direct connection between an adjacent artery and vein, the high blood flow from the artery through this direct connection causes the vein to become much larger and develop a thicker wall, much like an artery. In this manner, the A-V fistula thus provides a high blood-flow site for accessing the circulatory system and for performing hemodialysis.

Via this new arteriovenous blood flow connection, most blood will bypass the high flow resistance of the downstream capillary bed, thereby producing a dramatic increase in the blood flow rate through the fistula. Furthermore, although it is not medically feasible to repeatedly puncture an artery, formation of the fistula “arterializes” the vein. The arterialized vein can be punctured repeatedly, and the high blood flow permits high efficiency hemodialysis to occur.

Manner of Use

For each dialysis, two large-bore needles (normally 14-16 gauge) are inserted through the dialysis patient's skin and into the A-V fistula, one on the “arterial” end and the other on the “venous” end. When the tips of the needles are properly resting inside the access, a column of blood enters the end of tubing attached to each needle. Prior to beginning a dialysis treatment, a cap is removed from each tubing, thereby allowing blood to fill the tubing, and then a syringe of saline is injected through each tubing and needle. The two needles are then connected with rubber tubing to the inflow (arterial) and outflow (venous) lines of the dialysis machine, and dialysis is started.

The A-V fistula today is still considered to be the “gold standard” for vascular access. Because of its comparatively longer survival time and relatively lower level of major problems, it is the widely preferred choice of nephrologists. However, data from the 1997 U.S. Renal Data System Report indicates that only about 18% of all hemodialysis patients currently receive a primary A-V fistula; while about 50% of patients receive a PTFE graft (see below) and about 32% of patients receive a percutaneous catheter assembly at about two months time after starting hemodialysis therapy.

Recognized Problems

One of the main reasons that the A-V fistula is not widely used is that the surgically-created A-V fistula must “mature”. Maturation occurs when high pressure and high blood flow from the connected artery expand the downstream system of veins to which it is surgically connected. Surgeons have found that successful A-V fistula maturation is not possible in most hemodialysis patients because of the greatly increasing number of diabetic and older patients who have cardiovascular disease, which prevents the maturation process. Another reason for the low rate of usage is that since surgeons have failed so often to achieve fistula maturation after performing the costly A-V fistula surgery, the surgeon often will no longer even try this technique for creating a vascular access.

Another reason that A-V fistulas are relatively seldom used is that, even when fistula surgery is successful, the maturation of the constructed fistula generally takes approximately one to three months time to achieve. Since about half of all prospective patients have an immediate and urgent need to start hemodialysis as quickly as possible, the patient often cannot wait for A-V fistula maturation to occur. Thus critical patients must undergo costly temporary procedures and use percutaneous catheter assemblies to enable dialysis to take place, while waiting for maturation to occur.

In addition, it is one of the unfortunate drawbacks of A-V fistula, even with careful physical examination and/or the use of Doppler ultrasound or venography to identify suitable veins, that approximately 40-50% of patients do not have the vascular anatomy sufficient to create a primary A-V fistula. In addition, many dialysis veterans, for whom the use of an A-V fistula has previously failed, can no longer be considered as candidates for a primary A-V fistula.

Finally, it will be noted that a number of innovations and improvements in the making and use of A-V fistula have been proposed and technically developed. Merely exemplifying these developments are U.S. Pat. Nos. 6,669,709; 6,585,760; 6,398,764; 6,113,570; 5,830,224; and 4,822,341. Each of these issued patents, as well as their internally cited publications, is expressly incorporated by reference herein.

### The Prosthetic Graft

The typical prosthetic graft is a linear hollow cannula formed of a durable and biocompatible synthetic material. Currently, most surgeons consider polytetrafluoroethylene (hereinafter “PTFE”), a “TEFLON” type of material, to be the synthetic material of choice. Although the prosthetic graft is essentially structured to be a flexible linear tube, a varied range of differences and modifications in fibril length, wall thickness, external wraps, and ring supports, internal coatings in prosthesis size and shape have been developed; and the present commercial manufactures of PTFE hemodialysis grafts offer a variety of choices. See for example the variety of different PTFE graft structures which are commercially available and sold today—as listed by Table 1, page 21, in Scher L. A. and H. E. Katzman, “Alternative Graft Materials for Hemodialysis Access”, Sem Vasc Surg 17(1):19-24 (March, 2004).

When subcutaneously implanted by the surgeon, the PTFE prosthetic graft is integrally joined (by distal and proximal anastomoses) to a pre-chosen artery and a nearby vein in the arm; and thereby serves as a fluid flow connection and blood carrying bypass structure, which subsequently can be punctured by dialysis needle sets for vascular access and hemodialysis. Given the fact that A-V fistulas are largely not possible, a subcutaneously implanted PTFE prosthetic graft is today the most common form of permanent vascular access for the overwhelming majority of hemodialysis patients—because, in spite of the some severe limitations and risks for the conventionally known PTFE prosthetic graft, there simply is no better alternative available for them to date.

The usual locations for the subcutaneous insertion and anastomosis of a conventional PTFE prosthetic graft are typically in the forearm and the upper arm, and surgeons commonly use a PTFE prosthetic graft in either a loop or straight configuration. As a consequence, the choice of arterial blood vessels available for an inflow of blood into the PTFE prosthetic graft include the radial artery at the wrist, the antecubital brachial artery, the proximal brachial artery, the axillary artery, and rarely, the femoral artery. Similarly, the choice of venous blood vessel typically available for an outflow of blood from the PTFE prosthetic graft include the median antecubital vein, the proximal and distal cephalic veins, the basilic vein in the upper arm, the axillary vein, the jugular vein, and the femoral vein.

The Presently Existing Problems of PTFE Grafts

Despite these recent improvements and advances in prosthetic graft technology, the frequency of PTFE graft failure in-vivo remains very high. There are many reasons for failure of an implanted PTFE prosthetic graft, infection, and thrombosis and aneurysm formation being among them. However, the most common cause of failure by far is neointimal hyperplasia—as exemplified by the hyperplasia occurring at the venous side of the access graft anastomosis in an implanted prosthetic graft.

As shown by the photomicrograph of Prior Art FIG. 1 herein, neointimal hyperplasis results in the narrowing or “stenosis” of the distal outflow portion of the prosthetic graft device, and ultimately causes thrombosis of the entire length of the prosthetic graft, thereby rendering it unusable for dialysis. Although the thrombus can theoretically be removed, the underlying cause cannot; and thus the patient enters a spiral phase of recurrent failure, hospitalization and surgery. Despite innumerable attempts of various kinds over the years to prevent this particular cause of graft thrombosis and secondary failure, there have been few substantive advances to date.

Clearly therefore, the major disadvantages of the implanted PTFE prosthetic grafts are stenosis (i.e., closing of the lumen) and thrombosis (i.e., clotting), both of which block the flow of blood. This dysfunction occurs in almost all graft patients several times during their lives; and, because it interferes with life-sustaining dialysis, must be corrected quickly. Presently used interventional procedures include angioplasty to open the stenosis and infusion of thrombolytic agents such as urokinase to dissolve the clots. Also, various clinical studies report that the mean time for the operational use of the PTFE graft progressively decreases after each such corrective procedure; and such progressive decreases continue until the operational time is so short that the surgeon has little choice except to replace the graft. It is particularly noted that the survival time of the conventional PTFE graft, including all repairs necessary to maintain its function, currently averages only about two years.

Medical interventions to maintain PTFE prosthetic grafts and to treat patient complications (infection, thrombosis and aneurysm formation) are also expensive. Furthermore, declotting of the prosthetic graft is required every nine months or so on average. Also, because only three anatomic sites exist in each human arm for the placement of the prosthetic graft, the current medical practice is to perform additional screening procedures in an attempt to extend the survival time of the graft. Although these additional procedures add cost and inconvenience, they have yet to improve significantly the mean time interval between interventional repairs, although they may in fact improve the prosthetic graft survival life as such.

### Overview

In short, there remains a long standing and well recognized need for substantive improvements in prosthetic graft constructs and the manner of their surgical implantation subcutaneously. Moreover, a major clinical imperative exists today to find a more effective means for avoiding stenosis and thrombosis in the implanted prosthetic grafts as well as to reduce the frequency of the interventional repairs. Accordingly, were such improvements to be developed, the innovation would be recognized and accepted by medical practitioners and surgeons alike as being an unexpected development which provides major benefits and unforeseen advantages for the hemodialysis patient.

### SUMMARY OF THE INVENTION

The present invention has multiple aspects.

A first aspect of the invention provides a surgical prosthetic endograft insertion kit whose components are used by a surgeon to create a durable vascular access suitable for long-term hemodialysis in a particular subject afflicted with end stage renal disease, said surgical prosthetic endograft insertion kit comprising:

(a) a subject-customized prosthetic endograft suitable for the carrying of flowing blood, which is configured as a flexible, elongated hollow tube and is constructed of at least one durable and biocompatible material, said prosthetic graft article comprising
(i) a hollow ribbed medial section having a predetermined length, external diameter size, tubular wall thickness, and internal lumen diameter, and whose tubular wall can be repeatedly penetrated on-demand by dialysis needles,
(ii) a hollow distal conduit arm having two open ends, one end terminating as a discrete distal conduit end and the other end being integrally joined to and in fluid flow communication with said ribbed medial section, said distal conduit arm being of predetermined external diameter size, tubular wall thickness, and internal lumen diameter, and having a subject-customized linear length which is to be custom-sized by a surgeon such that after in-vivo insertion of said sized distal conduit arm into a pre-chosen vein in the particular subject, said distal conduit end will float freely within the vein and anatomically lie adjacent to the cavo-atrial junction of the heart in the particular subject,
(iii) a hollow proximal conduit arm having two open ends, one end terminating as a discrete proximal conduit end and the other end being integrally joined to and in fluid flow communication with said ribbed medial section, said proximal conduit arm being of predetermined-external diameter size, tubular wall thickness, and internal lumen diameter, and having a subject-customized linear length which is to be custom-sized by a surgeon such that said sized proximal conduit arm can be subcutaneously positioned over its entire sized length within the upper limb in a particular subject, and said proximal conduit end can be surgically joined to and anastomosed at a pre-selected anatomic site with a pre-chosen artery in the upper limb of the particular subject;

(b) a vascular balloon catheter formed of durable material and having pre-set dimensions, said vascular balloon catheter comprising a at least one substantially tubular stand having an internal lumen, an access port joined to one end of said tubular strand, and an inflatable and deflatable on-demand balloon disposed at the other end of said tubular strand, wherein said vascular balloon catheter serves as an obturator for said prosthetic endograft and is able to accommodate said distal conduit arm of said endograft over said balloon to form a coupled assembly;

(c) a tunneling obturator system comprising at least one elongated obturator of fixed dimensions and configuration having a conically-shaped tip end and which can be employed to form a subcutaneous tunnel passageway within the tissues of the body; and

(d) Seldinger technique workpieces comprising

a Seldinger needle of specific gauge,

a series of graded vascular dilators of known linear length and diameter which can be threaded over a guide wire, and

a guide wire of specified thickness and length.

A second aspect of the invention provides a surgical method for creating a durable vascular access suitable for long-term hemodialysis in a living subject afflicted with end stage renal disease, said surgical method comprising the steps of:

(1) creating a first insertion site at a pre-selected anatomic position in the neck/shoulder of the living subject to percutaneously puncture a pre-chosen vein;

(2) preparing a subject-customized prosthetic endograft configured as a flexible, elongated hollow tube and constructed of at least one durable and biocompatible material, said prosthetic endograft comprising
(i) a hollow ribbed medial section having a predetermined length, external diameter size, tubular wall thickness, and internal lumen diameter, and whose tubular wall can be repeatedly penetrated on-demand by dialysis needles,
(ii) a hollow distal conduit arm having two open ends, one end terminating as a discrete distal conduit end and the other end being integrally joined to and in fluid flow communication with said ribbed medial section, said distal conduit arm being of predetermined external diameter size, tubular wall thickness, and internal lumen diameter, and having a subject-customized linear length which is custom-sized by the surgeon such that after in-vivo insertion of said sized distal conduit arm into a pre-chosen vein in the particular subject, said distal conduit end will float freely within the vein and anatomically lie adjacent to the cavo-atrial junction of the heart in the particular subject,
(iii) a hollow proximal conduit arm having two open ends, one end terminating as a discrete proximal conduit end and the other end being integrally joined to and in fluid flow communication with said ribbed medial section, said proximal conduit arm being of predetermined external diameter size, tubular wall thickness, and internal lumen diameter, and having a subject-customized linear length which is custom-sized by the surgeon such that said sized proximal conduit arm can be subcutaneously positioned over its entire sized length within the upper limb in a particular subject, and said proximal conduit end can be surgically joined to and anastomosed at a pre-selected anatomic site with a pre-chosen artery in the upper limb of the particular subject;

(3) procuring a vascular balloon catheter formed of durable material and having pre-set dimensions, said vascular balloon catheter comprising at least one substantially tubular strand having an internal lumen, an access port joined to one end of said tubular strand, and an inflatable and deflatable on-demand balloon disposed at the other end of said tubular strand;

(4) passing said prosthetic endograft over said vascular balloon catheter such that said distal conduit arm of said prosthetic endograft is placed over said balloon of said vascular catheter, and then inflating said balloon on-demand to form a coupled assembly;

(5) percutaneously passing said coupled assembly through said first insertion site at a pre-selected anatomic position into the internal channel of the pre-chosen vein in the living subject, whereby said distal conduit arm of said coupled assembly comes to rest entirely within the channel of the pre-chosen vein, and whereby said distal conduit arm end floats freely and anatomically lies within the pre-chosen vein adjacent to the cavo-atrial junction of the heart in the living subject;

(6) deflating said balloon of said vascular balloon catheter on-demand to release said anatomically positioned distal conduit arm of said prosthetic endograft from said coupled assembly, and then removing said vascular balloon catheter from the vein without physically displacing said anatomically positioned distal conduit arm;

(7) creating a second insertion site at a second pre-selected anatomic position in the upper limb of the particular subject to gain access to a pre-chosen artery in the upper limb of the particular subject;

(8) mobilizing a segment of the accessed pre-chosen artery in the upper limb of the particular subject;

(9) surgically forming a subcutaneous tunnel passageway within the upper limb and which extends upwardly from said second insertion site and terminates adjacent to the first insertion site in the neck/shoulder of the particular patient, said formed subcutaneous tunnel and open passageway being substantially parallel to the anatomic location of the pre-chosen artery within the upper limb;

(10) passing said proximal conduit arm of said prosthetic endograft into and through the length of said subcutaneous tunnel and open passageway such that said custom-sized proximal conduit end lies adjacent to said second insertion site on the upper limb of the particular patient;

(11) introducing said ribbed medial section of said prosthetic endograft through said first insertion site such said ribbed medial section lies subcutaneously adjacent to said open passageway and subcutaneous tunnel; and

(12) joining and anastomosing said custom-sized proximal conduit end to said mobilized segment of the pre-chosen artery in the upper limb of the particular subject; and

(13) surgically closing said first and second insertion sites.

### BRIEF DESCRIPTION OF THE FIGURES

The present invention may be more easily understood and better appreciated when taken in conjunction with the accompanying Drawing, in which:

Prior Art FIG. 1 is a photomicrograph showing neointimal hyperplasis, a medical condition which results in the narrowing (or “stenosis”) of the distal outflow portion of a conventionally known PTFE graft;

FIG. 2 diagrammatically illustrates a preferred embodiment of the prosthetic endograft in the present invention;

FIG. 3 is a photograph showing a manufactured preferred embodiment of the endograft obturator in the present invention;

FIGS. 4A and 4B diagrammatically illustrate a preferred embodiment of vascular balloon catheter employed as an obturator in the present invention;

FIG. 5 is a photograph showing a manufactured preferred embodiment of the vascular balloon catheter in the present invention;

FIG. 6 is a photograph showing the portal access end of the manufactured vascular balloon catheter of FIG. 5;

FIG. 7 is a photograph showing the balloon in the manufactured vascular balloon catheter of FIG. 5;

FIG. 8 diagrammatically illustrates the combined assembly of the endograft obturator of FIG. 2 in relationship to the vascular balloon catheter of FIG. 5;

FIG. 9 is a photograph showing the combined assembly of the endograft obturator of FIG. 2 in relationship to the vascular balloon catheter of FIG. 5 as manufactured embodiments;

FIG. 10 is a photograph showing details of the relationship between the balloon of the vascular balloon catheter and the distal conduit arm of the endograft as a combined assembly in the manufactured embodiment of FIG. 9;

FIG. 11 illustrates a preferred obturator used as the tunneling apparatus to form a typical vascular access;

FIG. 12 illustrates the conically-shaped, distal end tip in the obturator of FIG. 11;

FIG. 13 is a photograph showing a tangible embodiment of the preferred elongated obturator useful for forming a subcutaneous tunnel passageway in-vivo;

FIG. 14 is a photograph showing a preferred embodiment of the complete surgical insertion kit of the present invention;

FIGS. 15A-15F illustrate the steps of the modified Seldinger technique;

FIG. 16 illustrates the anatomic positioning of the major arteries existing within the human arm;

FIG. 17 illustrates the anatomic positioning of the major veins existing within the human body;

FIG. 18 diagrammatically illustrates the insertion of a guide wire and a radiographic sheath extended through the internal jugular vein into the right atrium of the human heart;

FIG. 19 illustrates the insertion of a endograft and vascular balloon catheter in combined assembly over a guide wire through the internal jugular vein as well as the precise placement of the end of the distal conduit arm of the endograft at the cavo-atrial junction of the heart;

FIG. 20 illustrates the location of the subcutaneous tunnel passageway created in the upper arm;

FIG. 21 illustrates the placement of the subcutaneous tunnel passageway created in the upper arm of FIG. 16; and

FIG. 22 illustrates the proper internal positioning of the endograft as a whole within the human body as a durable vascular access.

### DETAILED DESCRIPTION OF THE PRESENT INVENTION

The subject matter as a whole which is the present invention provides a prosthetic endograft article, a modified surgical insertion kit, and an improved hybrid surgical insertion technique for creating an arteriovenous access in-vivo for hemodialysis. As a consequence, the present invention is able to prevent a primary cause of arteriovenous graft thrombosis; and provides a novel vascular access construction for successful long term use in maintenance hemodialysis.

The present invention employs a prosthetic endograft which is patient-customized by the surgeon as an endovascular component; and utilizes an improved and completely unique surgical method for endovascular insertion of the prosthetic endograft in a manner which does not require a distal anastomosis of the endograft. This technique allows the distal outflow end of the implanted arteriovenous access to remain unattached and freely floating within the internal lumen of a pre-chosen vein, which lies adjacent to and becomes joined with the heart.

The present invention is therefore able to provide a range of unforeseen advantages and unexpected medical benefits for the patient suffering from end stage renal disease. Among the unique advantages and significant medical benefits are the following:

(i) The present invention uses an endovascular approach to create a suture-less venous connection between the prosthetic endograft and the venous blood circulation of the patient's body. By definition, the term “endovascular” as used herein means the application of devices and/or methods within an existing blood vessel, usually percutaneously, in order to manipulate and employ the anatomy of the blood vessel itself. Accordingly, the term “endograft” as used herein identifies the unique prosthetic graft article provided by the present invention which is to be operative and functional as an arteriovenous access after its implantation into the patient's blood vessels and circulatory system in-vivo.

(ii) The present invention employs an adaptation and modification of the endovascular surgical procedure commonly known as the “elephant trunk” technique to insert a prosthetic graft article and join the article to a pre-chosen artery and vein. As a major outcome of using this modified surgical protocol, there is no anatomic anastomosis as such between the distal end of the prosthetic article and the venous blood circulation of the patient.

(iii) The absence of a distal anastomosis between the implanted prosthetic graft article and the venous circulation adjacent the heart negates all pathological flow dynamics at their point of common contact and juncture. This negation in pathological flow dynamics, in turn, will avoid and obviate the initiation and generation of neo-intimal hyperplasia at the distal end of the endovascular prosthetic article, then operative as the implanted arteriovenous access—such neo-intimal hyperplasia being recognized as being the most prevalent cause of vascular thrombosis. Accordingly, via this series of medical avoidances, the in-vivo occurrence of neo-intimal hyperplasia will be substantially eliminated and the incidence of vascular thrombosis will become markedly reduced.

(iv) The patency rates of the implanted endograft functioning as an arteriovenous access will be significantly greater than ever before, thereby reducing the severity of problems encountered after insertion and markedly increasing the duration and effective life of the implanted prosthetic article being used for hemodialysis. As a direct consequence and outcome, the morbidity and mortality rates for the patients using such an implanted vascular access for the performance of maintenance hemodialysis will become substantially reduced.

### I. The Conceptual Origins of the Present Invention

Endovascular surgery encompasses those conventionally known medical procedures whereby a therapeutic device is placed intraluminally—i.e., within the internal lumen of an existing blood vessel—using minimally invasive or percutaneous surgical techniques. However, endovascular surgery protocols have heretofore been used only to manage the pathology of the blood vessel itself; and have not ever before been used for the particular purpose of creating a durable vascular assess in-vivo for subsequently performing hemodialysis on a routine and regular schedule. Thus, while the technology and protocols for using endovascular surgery are themselves mature, this medical knowledge and skill has always been severely restricted in its actual applications towards permanent hemodialysis access.

The subject matter as a whole which comprises the present invention is based upon a thorough understanding and utilization of conventional endovascular surgical protocols; but constitutes a major adaptation and substantive alteration of previously existing surgical knowledge for an entirely new and different application; and employs a unique and meaningful modification of established surgical techniques for the express purpose of creating a permanent vascular access in-vivo which is suitable for the subsequent performance of hemodialysis. In particular, the present invention incorporates a combination of widely used open and percutaneous vascular surgery techniques with an endovascular component; and specifically utilizes a newly structured prosthetic endograft and its associated implantation methodology and equipment. The structural components of the implanted prosthetic device, as well as the manner of their surgical implantation into the body of a living patient, are therefore original, unique, and unforeseen in their clinical application and medical result.

The traditional and conventionally known technique—which has been adapted and substantively modified by the present invention—utilizes a concept popularized over a decade ago by Drs. Hans Borst and E Stanley Crawford known as the “elephant trunk” technique. The details of this conventional endovascular technique are given by Borst et al., “Extensive aortic replacement using ‘elephant trunk’ prosthesis”, Thorac Cardiovasc Surg 31:37-40 (1983); and Borst et al., “Treatment of aortic aneurysms by a new mutli-stage approach”, J Thorac Cardiovasc Surg 95:11-13 (1988).

In effect, Borst et al. generated a set of surgical procedures specifically for repairing complex thoraco-abdominal aneurysms. In these repair procedures, these surgeons would invaginate a length of prosthetic graft material into the descending thoracic aorta as a temporary aid and first stage step; and then, as a second stage step and event, afterwards perform a full and complete repair of an existing complex multisegment aortic aneurysm. In this manner, therefore, in order to repair the existing aneurysm in the proximal aortic segments, these surgeons would first implant a portion of the prosthetic graft material into the descending aorta without distal fixation as a part of their initial procedure. Then, at a subsequent time and second stage event, a segment of the previously implanted prosthetic graft, then floating freely within the descending intrathoracic aorta, would be used and incorporated via a second vascular anastamosis (or several anastamoses) and another additional segment of prosthetic graft material as part of a completed aneurysm repair.

In short, the Borst et al. multiple stage repair concept thus was utilized, as a temporary measure and first stage surgical event, to implant a prosthetic graft intraluminally; and initially leave a freely floating end of a prosthetic graft segment within the aorta, but without performing a distal vascular anastamosis. Then, as the requisite second stage repair event and followup surgical procedure, the technique introduced intraluminally and joined a second additional segment of vascular graft material to the freely floating end of the previously implanted prosthetic graft segment as a distal vascular anastamosis; and thereby generated a complete aneurysm repair. This multiple stage surgical protocol created by Borst et al. has become the gold standard of medical treatment for repairing a complex aortic aneurysm.

Considerable medical literature has been published regarding the merits of the Borst and Stanley multiple stage surgical technique for repairing a complex aortic aneurysm. Merely illustrative and representative of these medical publications are the following: Kuki et al., “An alternative approach using long elephant trunk for extensive sortie aneurysm: Elephant trunk anastomosis at the base of the inominate artery”, Circ 106 (12, Suppl. 1):1253-1258 (Sep. 24, 2002); Safi et al., “Staged repair of extensive aortic aneurysms: morbidity and mortality in the elephant trunk technique”, Circulation 104(24):2938-2942 (Dec. 11, 2001); Zanetti, P. P., “Replacement of the entire thoracic aorta according to the reversed Elephant Trunk technique”, J Cardiovasc Surg 42(3):397-4002 (January, 2001); and Keiffer et al., “Treatment of aortic arch dissection using the elephant trunk technique”, Ann Vasc Surg. 14(6):612-619 (November, 2000).

### II. The Components of the Surgical Endograft Insertion Kit

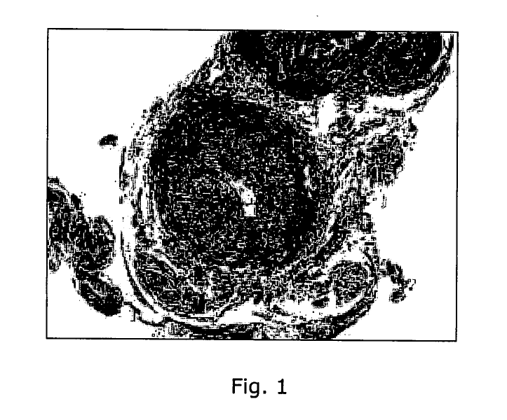

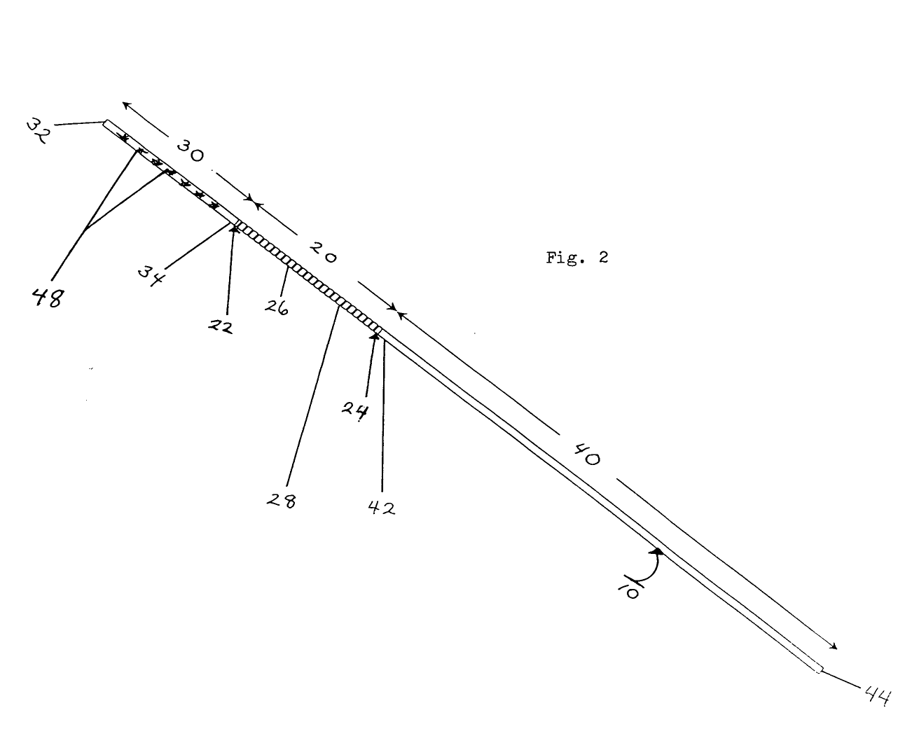

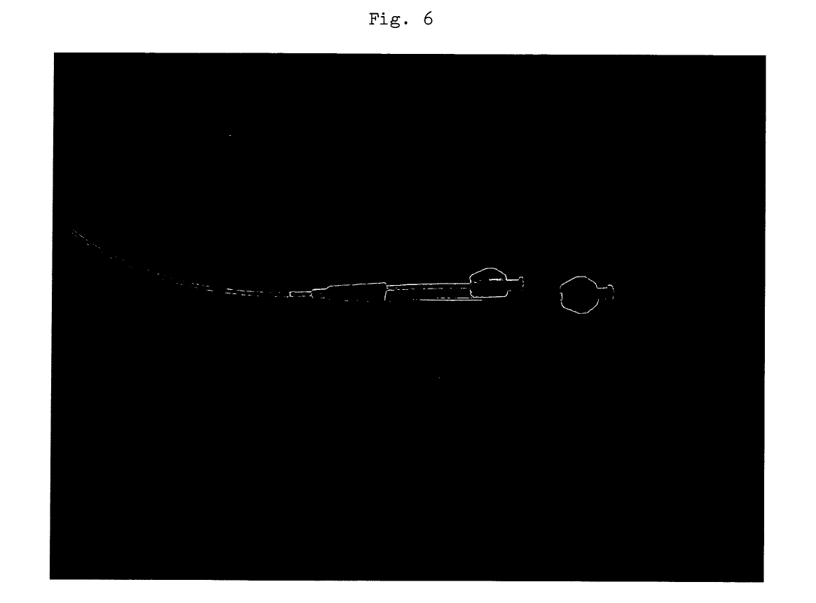

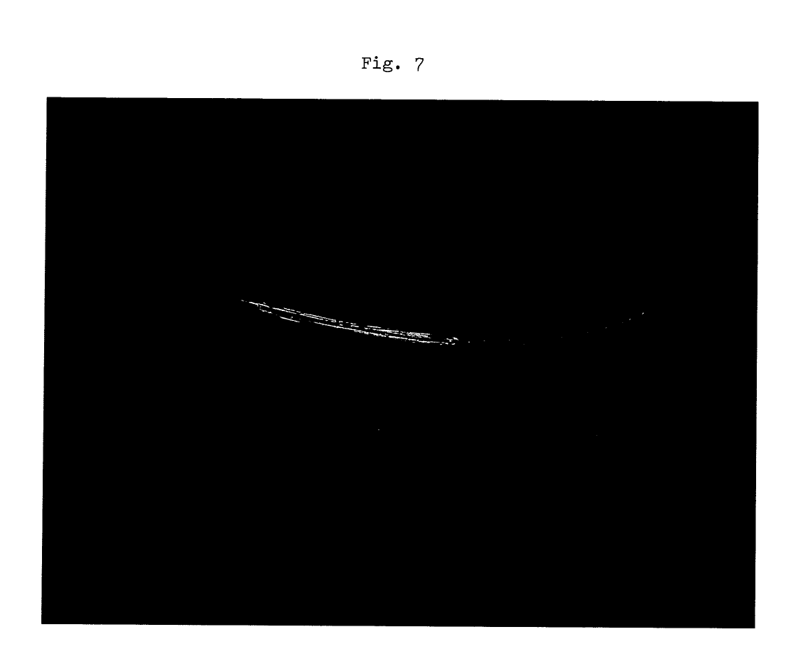

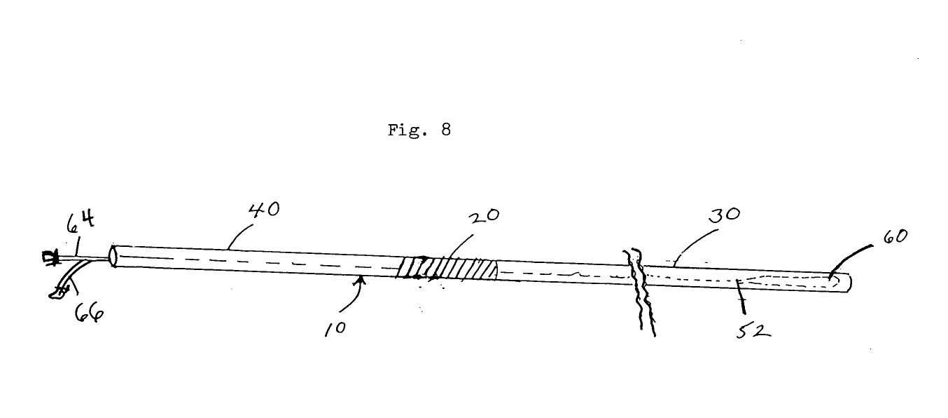

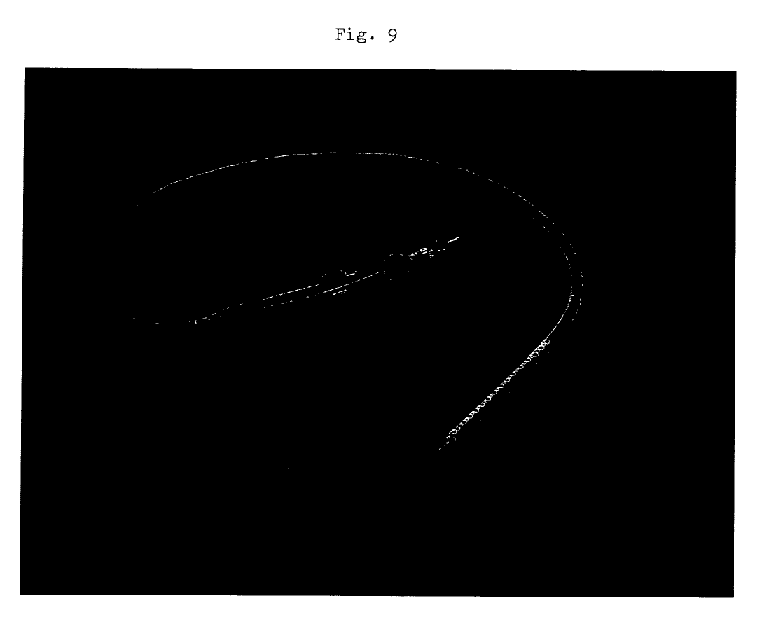

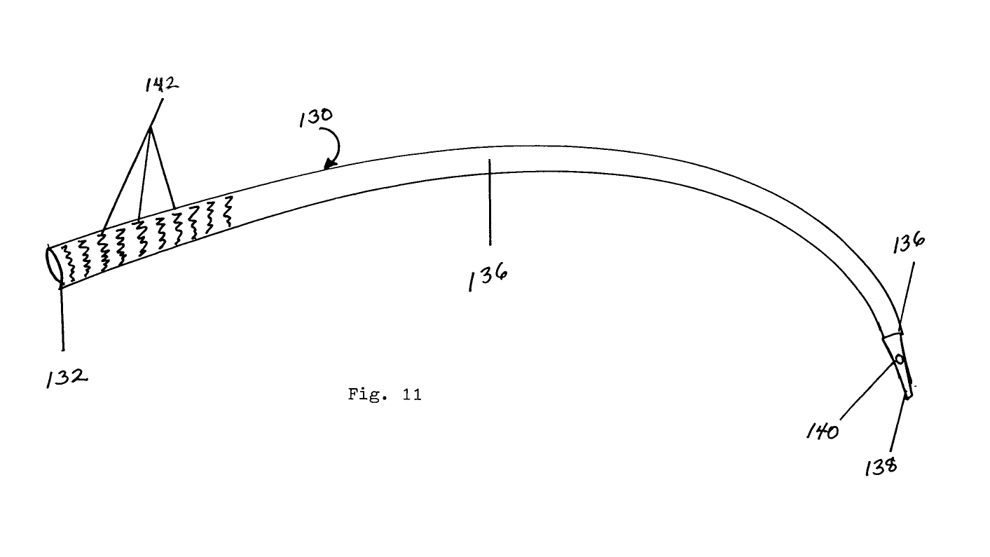

There are four article components which comprise the surgical insertion kit. These are: a prosthetic endograft (the graft article); an endograft (vascular graft) obturator; a tunneler system; and the Seldinger technique workpieces. Each of these components is described singly and in combination as a complete insertion kit in detail hereinafter, ready for intended use by a surgeon; and these components are illustrated individually and collectively by FIGS. 2-22 respectively.

### Component 1: The Prosthetic Graft Article (Endograft)

Desirably, the prosthetic endograft (or vascular graft article) is a pre-formed, flexible and elongated hollow tube structure which is manufactured in a variety of different linear lengths, alternative exterior diameter sizes, varying wall thicknesses, and differing inner lumen diameter sizes; and typically is composed of at least one durable and biocompatible material which may be entirely synthetic or be a derivative of living tissues. In addition, the durable material of the endograft structure offers a substantial flexibility for the inserted graft over the joints and anatomic bends in the body, and so prevents kinking of the endograft in-vivo.

In general, the pre-formed prosthetic endograft comprises three different structural component parts, as shown in detail by FIG. 2. These are: (i) the ribbed medial section; (ii) the distal conduit arm; and (iii) the proximal conduit arm.

(i) The ribbed medial section 20 of the endograft 10 illustrated by FIG. 2 is a hollow tube having two open ends 22. 24 as well as a predetermined length, external diameter size, tubular wall thickness, and internal lumen diameter. The circular tubular wall 26 of the ribbed medial section 20 is of a thickness and resilience which allows it to be repeatedly penetrated on-demand by dialysis needles whenever hemodialysis is to be performed. The ribs 28 are preferably disposed in a spiral pattern over the linear length of the medial section; and the ribs 28 serve as a structural reinforcement for the medial section over its intended long term of use.

(ii) The distal conduit arm 30 of the endograft 10 is a hollow tube having two open tubular ends 32, 34. One open end terminates as a discrete distal conduit end 32; while the other open end 34 is integrally joined to and lies in fluid flow communication with the open end 22 of the ribbed medial section 20. The distal conduit arm 30 is of predetermined external diameter size, tubular wall thickness, and internal lumen diameter. The distal conduit arm 30 also has an originally manufactured linear length which is to be shortened and custom-sized by a surgeon subsequently for the particular patient such that—after in-vivo insertion of the custom-sized distal conduit arm into a pre-chosen vein—the distal conduit end 32 will float freely within the internal lumen of the vein and anatomically lie adjacent to the cavo-atrial junction of the heart (but not actually within the atrium as such) within the particular subject. Preferably, there are a series of radiographic markers 48 along the linear length of the distal conduit arm 30 in each embodiment.

(iii) The proximal conduit arm 40 of the endograft 10 is a hollow linear tube having two open tubular ends 42, 44. One open end 42 terminates as a discrete proximal conduit end, while the other open end 44 is integrally joined to and in fluid flow communication with the open end 24 of the ribbed medial section 20. The proximal conduit arm 40 is a tubular segment of predetermined external diameter size, tubular wall thickness, and internal lumen diameter. The proximal conduit arm 40 also has an originally manufactured linear length which is intended to be shortened and custom-sized subsequently by the surgeon such that the sized proximal conduit arm can be subcutaneously positioned over its entire sized length within the upper limb of the particular subject in-vivo, and the proximal conduit end can be surgically joined to and anastomosed at a pre-selected anatomic site with a pre-chosen artery in the upper limb of the particular subject.

### A Preferred Embodiment

In the preferred embodiment of the endograft illustrated by FIG. 2, the prosthetic graft article is an elongated, hollow tubular structure of determinable length and has two discrete open ends and an internal lumen. Desirably, it is comprised of expanded polytetrafluoroethylene (or “E-PTFE”); is about fifty five (55) cm in overall linear length; and is about six (6) mm in outer diameter. However, the dimensions of the endograft may vary greatly among its different embodiments; and the total linear length of an endograft will typically vary from about 30-60 cm, while the exterior diameter of an endograft will typically vary in size from about 4-8 mm.

In a highly preferred expanded-PTFE embodiment, the endograft has a spiral ribbed medial section which typically is about fifteen to twenty (15-22) cm in length. This ribbed medial section is integrally joined to and is in fluid flow communication with a distal conduit arm and a proximal conduit arm. Preferably, the distal conduit arm of the endograft is a hollow tube, ranging from about twelve to fifteen (12-15) cm in length and terminates as a discrete distal (blood outflow) conduit end. Similarly, the preferred proximal conduit arm of the endograft is also a hollow tube, ranging from about fifteen to eighteen (15-18) cm in length and terminates as a discrete proximal (blood inflow) conduit end.

It is very desirable that each embodiment of the endograft include a series of radiographic markers disposed upon the exterior surface of the distal conduit arm at pre-measured distances and fixed intervals along its linear length up to the distal conduit end. These radiographic markers will typically be sub-millimeter sized titanium markings impregnated into the graft material itself, preferably at exactly one centimeter length distances. The markers will be visible both fluoroscopiclally and radiographically; be MRI (magnetic resonance imaging) compatible; and be used for measuring the exact distance and identifying the precise location of the distal conduit arm. In particular, these radiographic markers will provide an identifiable image of and visualization of the anatomic positioning for the distal conduit arm within the lumen of the pre-chosen vein; and permit accurate placement of the discrete distal conduit end such that it lies adjacent to the cavo-atrial junction of the heart (but not actually within the atrium as such) within the particular subject.

The Presently Existing Variety of PTFE Materials for Fabricating Endografts

A wide range and variety of different PTFE chemical formulations and compositions, methods of manufacture, and fabrication formats are commonly known and used today. Merely exemplifying the diversity of these PTFE materials and modes of fabrication are: The laminated self-sealing vascular access graft of U.S. Pat. No. 6,319,279; the PTFE vascular graft and method of manufacture described by U.S. Pat. No. 6,719,783; the dialysis graft system with self-sealing access ports disclosed by U.S. Pat. No. 6,261,257; and the self-sealing PTFE vascular graft and manufacturing methods recited by U.S. Pat. No. 6,428,571. In addition, a varied range of structural modifications differing in fibril length, wall thickness, external wraps, and ring supports, internal coatings in prosthesis size and shape are presently known. See for example U.S. Pat. Nos. 4,082,893; 4,177,334; 4,250,138; 4,304,010; 4,385,093; 4,478,898; 4,482,516; 4,743,480; 4,816,338; 4,478,898; 4,619,641; and 5,192,310. Accordingly, the text of each of these issued patents, as well as their internally cited publications, is expressly incorporated by reference herein.

Presently Available Alternative Biocompatible Materials for Fabricating Endografts

The biocompatible composition comprising the material substance of the prosthetic graft article, however, is not intended to be confined or to be limited to the use of PTFE (in any of its conventionally known chemical formulations). To the contrary, a range and variety of different and alternative graft materials are presently available. Among these alternative materials are:

(i) “DACRON” or polyethylene terephthalate fibers and fabrics which were used as one of the original materials for prosthetic grafts (U.S. Pat. No. 2,465,319 assigned to Dupont Chemical Corp.);

(ii) multi-layered and self-sealing polyurethane (manufactured by Thoratec, Pleasanton, Calif.); bioartificial matter derived from mesenteric vein (Hancock Jaffee Laboratories inc., Irvine, Calif.); and

(iii) a cryopreserved allograft material in which cellular elements have been removed using antigen reduction technology (CryoLife Inc., Kennesaw, Ga.).

Details and important considerations about these different and alternative graft compositions are described in Glickman, M. H., J Vasc Surg 34:45-472 (2001); Matsura et al., Ann Vasc Surg 14:50-55 (2003); Bolton et al., J Vasc Surg 36:464-468 (2002); and Scher, L. A. and H. E. Katzman, Sem Vasc Surg 17(1):19-24 (March, 2004).

### Component 2: The Endograft Obturator

The endograft obturator is a discrete structure used by the surgeon to carry, or to support, or to introduce the endograft prosthesis into the vascular system of the living patient. While there are various devices which can be used to perform an introduction of the endograft prosthesis described herein, the present methodology prefers to use a conventionally known angioplasty balloon catheter as the vascular obturator or carrier device of choice.

The Conventionally Known Angioplasty Balloon Catheter

Angioplasty balloon catheters are a class of medical therapeutic devices which are typically used to dilate an area of arterial blockage. Structurally, the conventional angioplasty balloon catheter has an inflatable small sausage-shaped bulb or balloon at its end tip, which can be inflated and deflated on-demand; and this capability is often utilized in the treatment of coronary artery disease. The particular medical technique which utilizes such angioplasty balloon catheters for this purpose is frequently called “Percutaneous Transluminal Coronary Angioplasty”, or PTCA.

In the treatment of coronary artery disease, the angioplasty or vascular balloon catheter is employed to open the channel of diseased arterial segments; to relieve the recurrence of chest pain; to increase the quality of life; and to reduce other complications of coronary disease. Procedurally, the angioplasty or vascular balloon catheter is introduced through a small hole in the skin at the groin, or sometimes the arm; and is placed in-vivo within an occluded blood vessel. The balloon is then inflated to open the artery and/or physically breakup the obstruction lying within the blood vessel. Since the medical technique is performed through a small needle-sized hole, this mode of treatment is much less invasive than open-body surgery; and the angioplasty balloon treatment can be repeatedly performed, should the patient later develop coronary disease in the same or another artery in the future.

Typically, prior to performing PTCA, the radiologist or cardiologist determines the anatomic location and type of blockage, as well as the shape and size of the coronary arteries. These determinations help the physician/cardiologist decide whether it is appropriate to proceed with angioplasty, or whether one should consider another form of treatment—such as stenting, atherectomy, medications, or excision surgery.

The Conventionally Available Kinds of Vascular Balloon Catheters

A diverse range of vascular (or angioplasty) balloon catheters have been structurally designed. The range and diversity of these articles and structures are well described in the patent literature. A representative, but non-exhaustive, listing is described by U.S. Pat. Nos. 4,456,011; 4,744,366; 4,763,654; 4,950,239; 5,041,090; 5,312,430; 6,132,824; 6,136,258; 6,231,588; 6,261,260; 6,689,152; and 6,805,898; and the references cited internally within these issued U.S. patents.

Similarly, a variety of diverse materials and alternative modes for construction of medically acceptable vascular balloon catheters is also conventionally known. A representative, but non-exhaustive, listing is provided by U.S. Pat. Nos. 4,429,062; 4,456,011; 4,477,255; 4,551,132; 5,500,180; 5,797,877; 6,086,556; 6,482,348; 6,805,898; 6,896,842; and 6,913,617, and the references cited internally within these issued U.S. patents.

The medical and commercial literature also provides many useful examples and instances of using vascular (or angioplasty) balloon catheters for therapeutic treatment. See for example: Currier J. and Faxon D., “Restenosis after PTCA: Have We Been Aiming at the Wrong Target?” J Am College Cardiology, 25(2):516-517 (1995); King, S., “The Role of New Technology in Balloon Angioplasty,” J Am Heart Assoc, 2(5):74-77 (1992); Schael, G., “Measuring Stiffness of Materials for Catheter Design,” Med Plast Biomat, 1(1):19 (1994); Serruys, T., Interventional Cardiology, Philadelphia, Current Medicine, pp 1.71.9 (1994)

A Preferred Embodiment of the Vascular Balloon Catheter Used as an Endograft Obturator

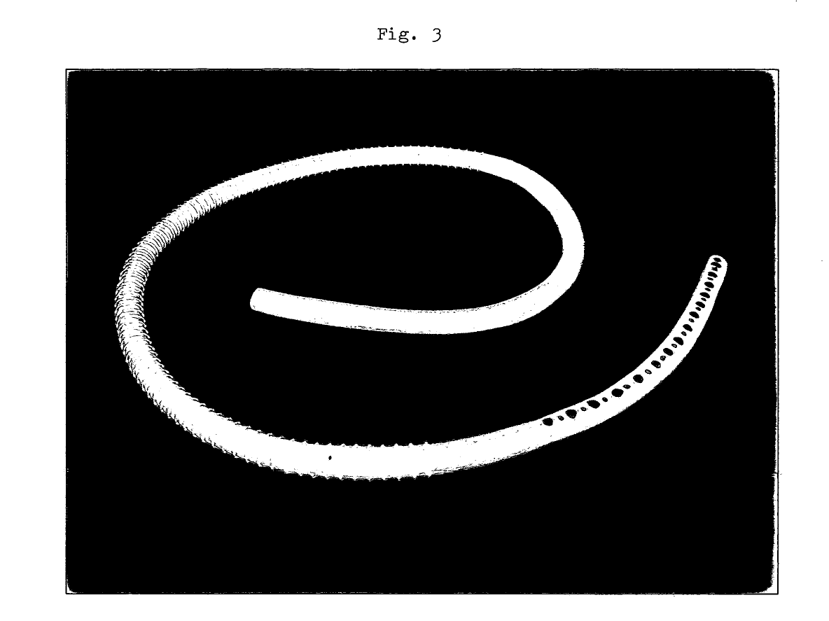

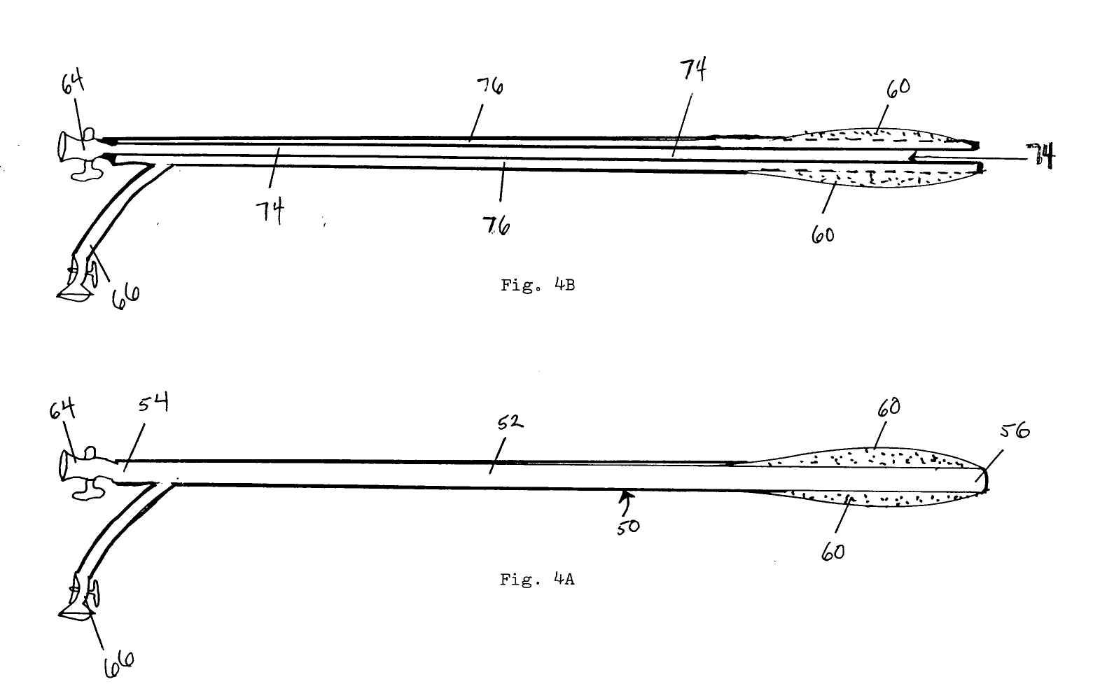

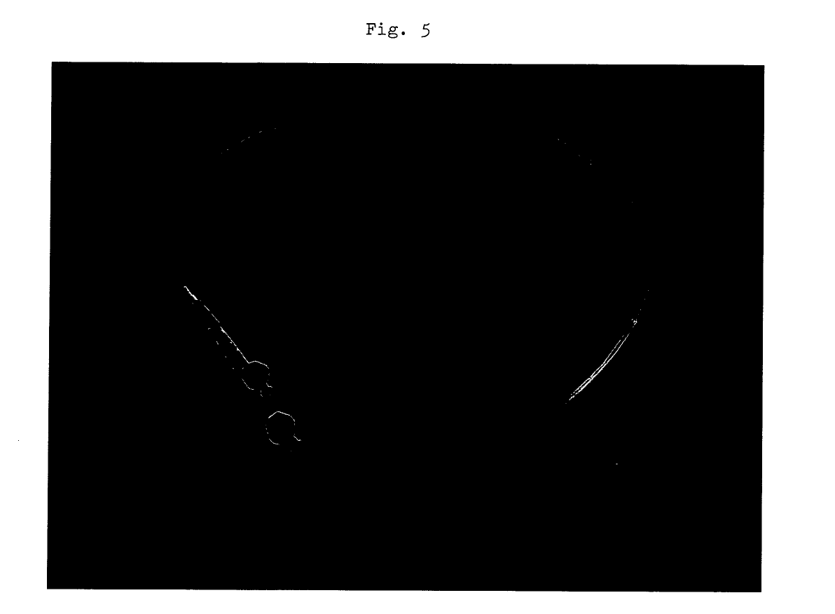

A preferred embodiment of the vascular (or angioplasty) balloon catheter structure is illustrated by FIGS. 4-7 respectively. As shown diagrammatically by FIGS. 4A and 4B and as manufactured embodiments by FIGS. 5-7 respectively, the preferred structure appears as a double-port and double-lumen vascular balloon catheter 50, having a substantially elongated tubular body 52, and typically measuring from 75-150 cm in overall length from the proximal end 54 to the distal end 56. In this preferred embodiment, the catheter body is formed as a double lumen tubular strand. The vascular catheter structure also includes an inflatable and deflatable on-demand (sausage-shaped) balloon 60, which is attached to the tubular body 52 and encompasses the distal end 56.

As shown, the vascular balloon catheter structure 50 provides two discrete access ports 64, 66—each of which is disposed adjacent the proximal end 54 of the catheter body 52. A first portal access is termed the proximal port 64 and is joined to a first tubular strand 74 having an elongated individual internal lumen. The second portal access is termed the distal port 66 and is joined to a second tubular strand 76 also having an elongated individual internal lumen—which, in this design, encompasses and surrounds the entirety of the first tubular strand 74 over most of the linear length of the catheter body 52.

The proximal access port 64 of the catheter 50 is a hollow conduit and extends over the linear length of the catheter body 52. The proximal port 64 is used for the introduction and passage of one or more guide wires (each preferably having a minimum 0.038 inch diameter) over the linear length of the catheter body; and also offers an entry portal for the instillation of a wide range of fluid agents through the tubular catheter body, such agents being exemplified by saline, blood, contrast medium, and the like.

In comparison, the distal access port 66 serves as the structural means for inflating and deflating the balloon 60 at will. The distal access port 66 will thus carry and convey fluids (gases or liquids) under limited pressure from an external source (not shown) to the balloon interior. In this manner, as more fluid is introduced via the distal access port into the balloon interior, the balloon volume will expand in ever larger degree; and conversely, when the fluid is released and removed from the interior of the balloon via the distal access port, the volume of the balloon will rapidly and markedly decrease.

In the embodiment shown by FIGS. 4-7 respectively, the vascular balloon catheter (serving as the endograft obturator) has a substantially double-lumen tubular wall formed of a hard, durable and biocompatible material such as polyurethane or polystyrene. The tubular configuration provides two separate and individual internal lumens of defined spatial volume for each tubular strand, one of which is attached to a discrete inflatable and deflatable on-demand balloon disposed at the distal end of the catheter body.

It will be recognized and appreciated that while a variety of balloon sizes (ranging from 5-8 mm in diameter and from 4-10 cm in linear length) may be used in the vascular catheter, it is highly preferred the balloon be about 10 cm in linear length after being inflated. Furthermore, the overall diameter of the sausage-shaped balloon tip (after full inflation) should be chosen and pre-sized to be approximately one millimeter (1 mm) larger than the inner (internal) lumen diameter of the endograft prosthesis—so that the balloon (when properly inflated) will be able to engage, support and carry the endograft into the patient's vascular system without being dislodged.

Functionally, for purposes of the present invention, the vascular (or angioplasty) balloon catheter serves as an obturator; and is employed to properly place and anatomically position the distal conduit arm of the endograft prosthesis within the superior vena cava of the patient. This function is utilized after the patient's venous system has been surgically accessed by needle puncture, and a guide wire placed therein for standard Seldinger technique catheter exchange.

The Combination of the Endograft and Vascular Balloon Catheter as a Coupled Assembly

The juncture and relationship of the endograft and the vascular balloon catheter is illustrated by FIGS. 8-10. As shown therein, it is intended that the endograft prosthesis 10 (described previously above) be placed over the vascular (or angioplasty) balloon catheter 50; and that the end of the distal conduit arm 30 of the endograft 10 then be extended over the axial length of the catheter such that the endograft distal arm 30 (having pre-calibrated radiographic markers) comes to rest and lies directly over the length of the deflated balloon 60. This combination and physical joining of the endograft and the vascular balloon catheter forms a coupled assembly and a combined unit, which is then employed as a joined entity in-vivo.

Once the distal conduit arm 30 of the endograft is properly positioned and lies disposed around the linear length of the deflated balloon 60, the balloon will then be inflated on-demand by the surgeon via the distal access port 66 to such a degree that the expanded balloon makes physical contact with and forms a fluid-tight fit and seal with the solid wall of the distal conduit arm of the endograft then disposed over and around the balloon.

After the balloon has been inflated, the volumetric tip of the inflated balloon will preferably extend about 0.5-1.0 cm beyond the end of the distal conduit arm 30 of the endograft; and the inflated balloon will present a tight, secure attachment and coupling with the interior surface of the endograft conduit arm wall—such that the inflated balloon will not subsequently disengage when the endograft-catheter coupled assembly is introduced into the vascular system of the patient.

The Intended Manner of Use for the Vascular Balloon Catheter as an Obturator

For in-vivo use, the surgeon will previously have made a percutaneous puncture site in the neck of the patient; which has then been enlarged and serves as the entry site where an angiographic dilator catheter is introduced and a guide wire is passed to the level of the cavo-atrial junction of the patient's heart. Serial angiographic dilators of graded caliber are used to enlarge the percutaneous entry site for the endograft-catheter coupled assembly as it is passed through the skin entry site (over an implanted guide wire) into the venous system of the patient. It is especially important that the endograft-balloon catheter coupled assembly can pass freely and easily as a combined unit through the open space of the puncture site and into the venous system of the living patient, in order that the coupled assembly then be placed radiographically into proper anatomic position in-vivo at the caval-atrial junction.

Accordingly, after proper dilation the endograft-balloon catheter combined unit is pushed through the skin entry site; is passed via the angiographic dilator catheter over a guide wire through the internal jugular vein and into the superior vena cava; and then is radiographically placed such that the distal arm end of the endograft lies precisely at the caval-atrial junction. The proximal port of the catheter is be used for instillation of contrast agent and to confirm proper anatomic position radiographically for the endograft.

Assuring good and proper anatomic positioning, the balloon can now be deflated by the surgeon; and the angioplasty catheter (obturator) then be separated, retracted, and completely removed from the endograft. As this surgical maneuver is performed, the patient's blood will flow in retrograde fashion from the right atrium into the interior of the distal conduit arm, up into and through the ribbed medial section of the endograft, and then flow out the proximal conduit arm end of the endograft. This blood flow will additionally confirm that a proper intravascular placement of the endograft has been achieved in-vivo. The proximal conduit arm of the endograft can now be occluded either by digital pressure or by using a standard vascular bulldog clip to prevent a meaningful loss of blood through the endograft.

### Component 3: The Tunneler Apparatus & Tunneling System

The complete insertion kit of the present invention also provides tangible means for forming a tunnel passageway subcutaneously within the soft tissues in the upper arm of a living human patient. Preferably, the tangible tunneling means comprises a unique single piece obturator of predetermined length and diameter, and which presents several distinct and unique structural features which aid in the formation of a subcutaneous open passageway for internal placement of the endograft.

Conventionally Available Tunneling Apparatus and Systems

It will be recognized and appreciated that the surgical implantation of the endograft is to be made subcutaneously within the soft tissues beneath the skin of the patient; and that when the in-vivo surgical procedure is completed, there are no structural elements or portions of the implanted prosthetic endograft that are visible or remain exposed on the exterior surface of the patient's skin.

To achieve the desired implantation, a tunnel passageway must be created subcutaneously in-vivo; and a variety of surgical tunneler methods and tunneling devices are presently known and commercially available for this purpose. Merely illustrating and representative of the currently available tunneling devices and tunneling methods are those described by U.S. Pat. Nos. 5,306,240; 4,832,687; 4,574,806; and 4,453,928. The text of each of these issued patents, as well as their internally cited publications, is expressly incorporated by reference herein. Any of these conventionally available devices and systems can serve as the means for forming a tunnel passageway subcutaneously within the soft tissues beneath the skin of the patient.

Tunneling Apparatus and Systems in General

As conventionally well established in the medical arts, a tunneling apparatus typically is a two-part system comprised of a tunnel sheath and a tunnel obturator. Both parts can be made of a material like polyethylene or polyurethane or polystyrene; and each part has sufficient structural rigidity to be passed into and through the subcutaneous tissue of a patient in-vivo in order that a tunnel passageway may be made in-situ. For purposes of the present invention, a preferred tunneling apparatus is illustrated by FIGS. 11-13 respectively.

A Preferred Tunnel Obturator

A preferred tunnel obturator 130 is illustrated by FIGS. 11, 12 and 13 respectively. As seen therein, the obtrurator 130 is an elongated solid rod approximately 30 cm in length and 0.8 mm in its largest diameter. The form of the obturator 130 typically has a thicker proximal end 132, when then ends to thin slightly in diameter over most of its axial body length 134, and also presents a narrowed, conically-shaped tip 138 at the distal end 136.

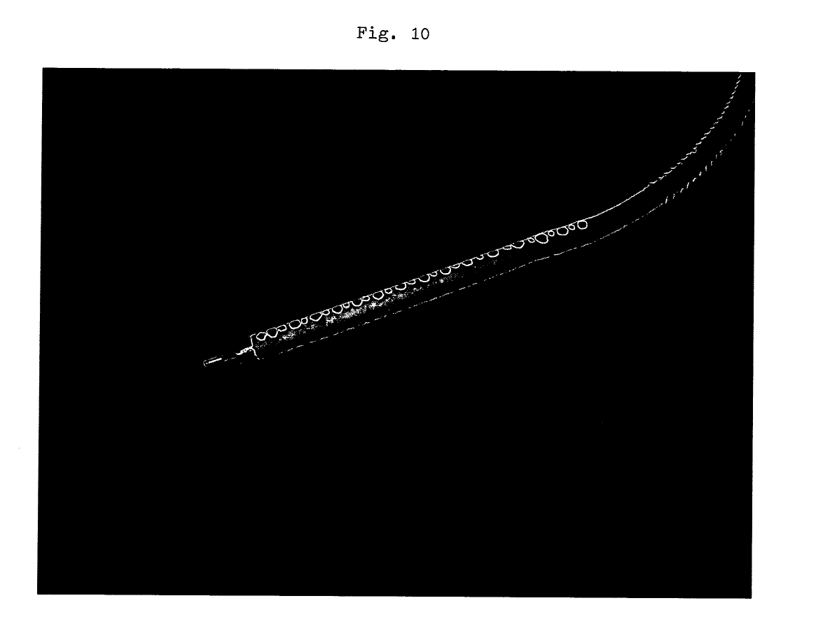

A distinct feature of the conically-shaped tip 138 existing at the distal end 136 is the presence of a preformed aperture (or hole) 140, which penetrates completely through the solid material substance of the obturator; and serves as an aid for the sutured securing of the endograft, as is described by the surgical method presented hereinafter. The intended location of the aperture 140 is shown in detail by FIG. 12.

It will be noted that the distal end 136 is formed as a bullet shaped tip which typically is about 1.5 cm in overall length; and includes a centrally located 0.3 cm segment having an aperture 140 lying within the diameter of the rod end, through which a suture can be passed. Following this centrally located segment is another tapering 1.0 cm linear segment of rod, which in turn, is followed by a 0.7 cm rod portion of uniform diameter. It will be appreciated also, that while the preferred lengths of each segment forming the conically-shaped tip 138 are presented, each segment length may be altered at will and vary markedly from those particulars given here, to meet particular use circumstances or the personal preference of the user.

In addition, as a highly desirable but completely optional feature, there are preferably a series of manufactured ridges 142 disposed over the exterior surface of the obturator 130 at the proximal end 132. These ridges 142 provide an improved grasping area or gripable handle for the obturator; and serve to aid in controlling the axial length of the obturator as it is pushed through the living tissues of the body.

It will also be recognized that several commonly used and conventionally known features are notably absent and missing from the structure of the obturator 130 shown by FIGS. 11-13 respectively: First, there is no central lumen as such and no internal cavity space at all within the elongated axial body length of the preferred obturator; rather, the entire axial length of the obturator—with the exception of the aperture 140 present within the conically-shaped end tip 138—is made of solid material. Thus, contrary to conventional tools, no guide wire or any other object of any kind can be passed axially through the obturator 130.

Second, there is no outer sheath as such, and no sheathing (nor tubing, nor catheter, nor outer covering) of any kind to be used in combination with the preferred obturator 130 as part of the process by which a tunnel passageway is subcutaneously formed in-vivo. Unlike many commonly used tunneling tools, the preferred obturator rod is employed alone and in isolation during the tunneling process.

Third, only human hand and arm generated, manual force applied at the grasping handle (proximal) end 132 is to be used in order to push the axial body length 134 and the conically-shaped distal end tip 136 of the obturator subcutaneously through the living tissues of the human body. The manufactured ridges 142 disposed over the exterior surface at the proximal end 132 thus function to aid the surgeon in controlling and guiding the direction of the obturator as the hand and arm generated force is applied by the surgeon to the proximal end of the obturator.

Intended In-Vivo Application and Usage

The tunneling obturator is intended to be used in-vivo after the endograft has exited the percutaneous skin site in the neck, and has been clamped to prevent bleeding and/or air from entering into the system. At that time, a subcutaneous tunnel will be made in order that the endograft can be placed in an in-line position subcutaneously down to the antecubital area of the arm.

To achieve these purposes, a small skin incision (approximately 2 cm in length) is made with a scalpel over the brachial artery (as it is palpated just above the elbow crease on the inner aspect of the arm). This incision is carried down only to the subcutaneous layer just below the skin, and lies above the muscle and fascia of the arm.

Grasping the proximal handle end of the solid rod obturator, the conically-shaped distal end tip is introduced into the incision just under the skin. While taking care to stay very superficial in the subcutaneous layer, the axial length of the obturator rod is passed up the arm in the direction of the neck—all the while staying in the same subcutaneous layer and using the natural rod-like curve of the obturator to guide the process and the progressive formation of the tunnel passageway. As the formed tunnel space anatomically reaches the area of the shoulder, the conically-shaped tip end of the obturator is aimed directly at the percutaneous exit site in the neck from where the endograft emerged. Again, this pathway should follow the natural curve of the tunneling device, which was engineered to fit the desired contours of the arm and create the preferred intended subcutaneous tunnel placement for the endograft.

Upon reaching the percutaneous puncture site in the neck (where the endograft exits and emerges), the obturator is maneuvered such that the conically-shaped distal end tip enters the pre-existing puncture site and thereby causes a merger of the newly formed subcutaneous tunnel with the percutaneous puncture site in the neck. Then, the surgeon threads the proximal conduit arm of the endograft over the conically-shaped tip end of the obturator; and extends the end of the proximal conduit arm over the obturator distal end tip for a distance of about 3 cm.

At this point a suture of any kind (but preferably a heavy ligature such as an O-silk suture) is passed through the conduit arm of the endograft and the aperture in the conically-shaped tip end; and then is tied circumferentially around the exterior surface of the conduit arm of the endograft. In this manner, the suture will secure both walls of the endograft conduit arm to the conically-shaped tip end of the obturator. Additional ties of suture (without using a needle) then are also preferably made to reinforce and further secure the endograft to the distal tip end of the obturator.

After the endograft is securely fastened to the conical tip end of the tunneler, the entire axial length of the obturator is then pulled rearward through the tunnel; and is withdrawn completely from the newly formed subcutaneous tunnel passageway at the second skin incision existing over the brachial artery, a withdrawing maneuver which concomitantly brings with it the proximal conduit arm of the endograft. Care is taken to be sure the endograft does not twist or kink during the withdrawl maneuver and that the endograft conduit arm slides smoothly through the newly created tunnel passageway. As the obturator-endograft connection exits through the incision over the brachial artery, the silk suture (securing the two together) will be cut, thereby releasing the endograft from the conically-shaped tip end of the obturator. The proximal arm of the endograft is then clearly visble at the incision over the brachial artery; can be physically grasped and manipulated by the surgeon; and may now be utilized for the arterial anastamosis.

### Component 4: The Seldinger Technique Workpieces

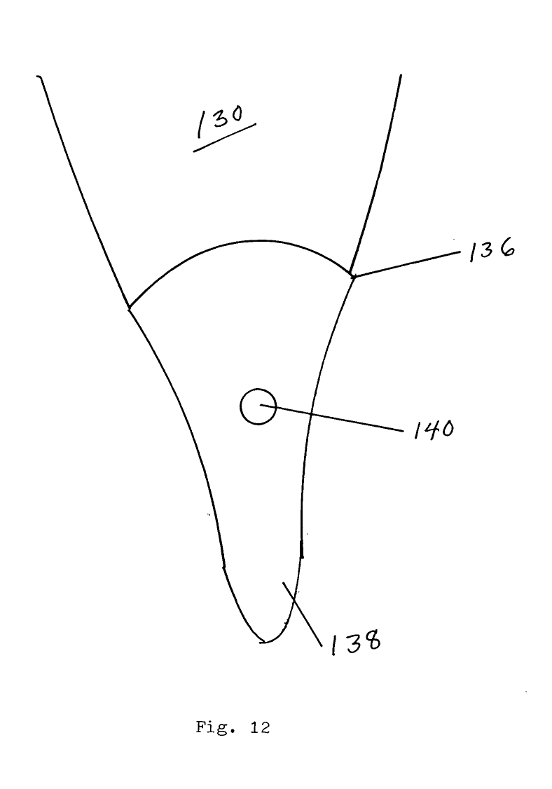

The Seldinger technique workpieces comprise a grouping which will typically include at least one thin-walled puncture needle 160 (preferably 18-22 gauge); a radiopaque vein dilator 170 (preferably 20-25 cm in linear length and typically of 5-6 French diameter size) which has a series of radiopaque (typically 1 cm sized) markers over its linear length; and at least one flexible guide wire 180 (preferably 0.038 inch thick and 100 cm in length). These items as a grouping are illustrated as individual component parts present within the complete insertion kit 200, as shown by FIG. 14.

The Modified Seldinger Technique:

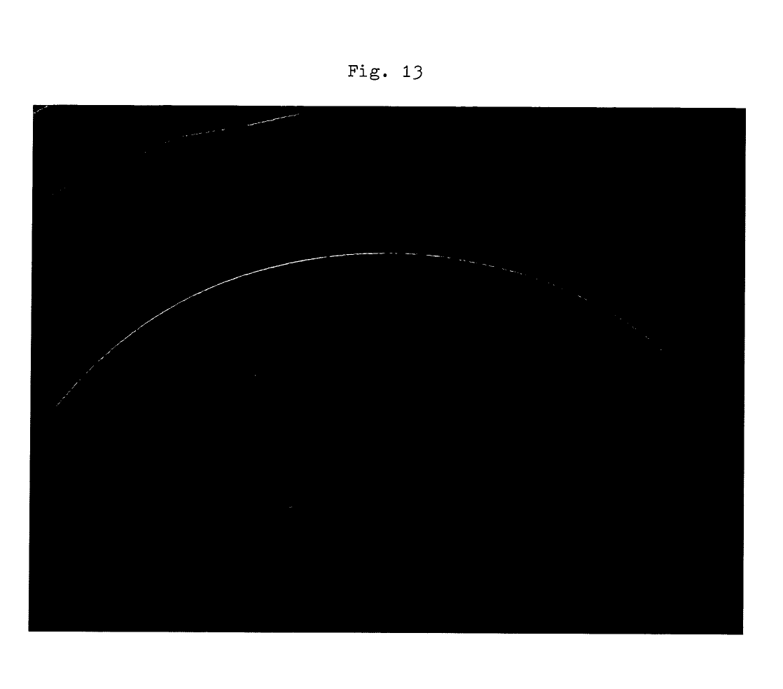

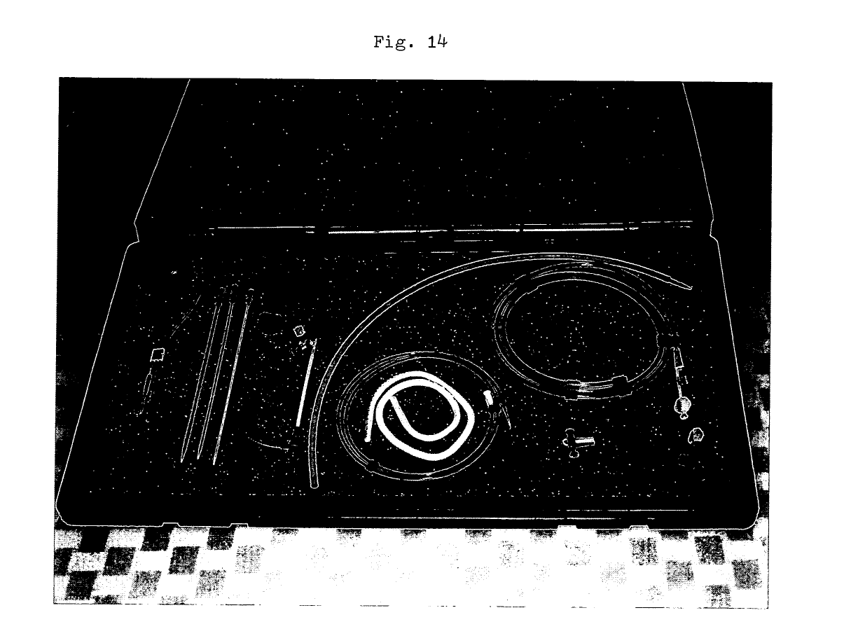

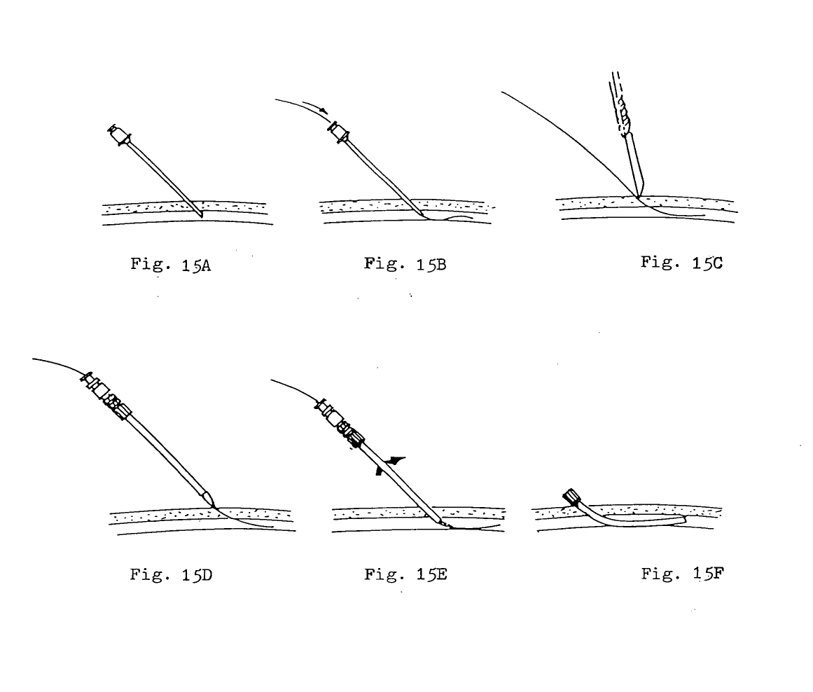

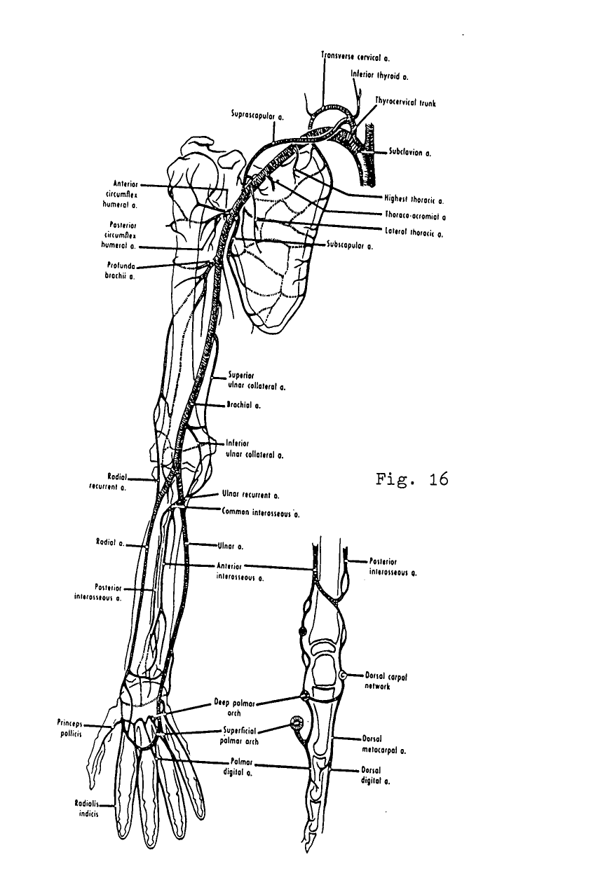

The percutaneous use of these workpieces is illustrated by the modified Seldinger technique which is shown by FIGS. 15A-15F respectively.

FIG. 15A shows a blood vessel being punctured with a small gauge needle, which has been percutaneously introduced through the epidermis and dermis by the surgeon. Once vigorous blood return occurs, a flexible guidewire is placed into the blood vessel via the bore of the needle as shown by FIG. 15B. The needle is then removed from the blood vessel, but the guidewire is left in place. Then the hole in the skin around the guidewire is enlarged with a scalpel as shown by FIG. 15C. Subsequently, a dilator-introducer sheath is placed over the guidewire as shown by FIG. 15D. Thereafter, the sheath and dilator is advanced over the guidewire and directly into the blood vessel as shown by FIG. 15E. Finally, the dilator and guidewire is removed while the sheath remains in the blood vessel, as illustrated by FIG. 15F. Certain diagnostics, contrast enhanced imaging and anatomic confirmation will be performed using the introducer-sheath and side arm port.

### III. Anatomic Considerations

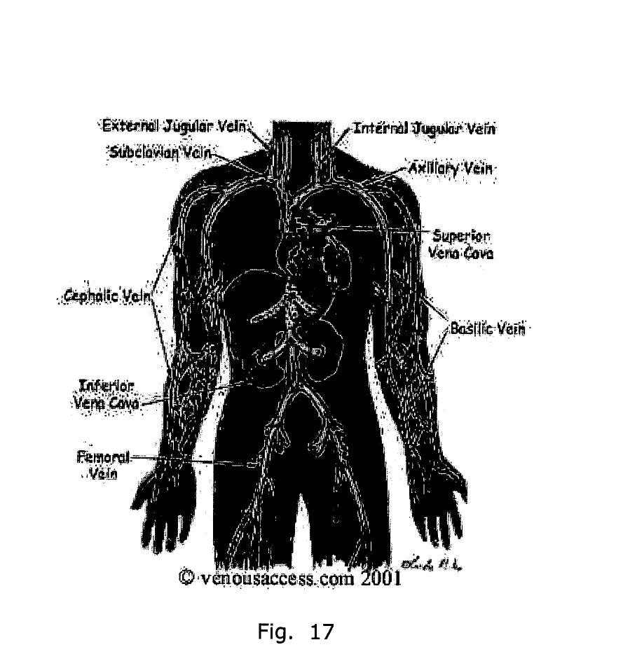

Clearly, the surgeon has a choice of which vein and which artery shall be employed and is to be connected for blood carrying purposes via the prosthetic graft article and surgical methodology of the present invention. While somewhat limited in his selection of suitable blood vessels by the anatomy of the human body, the surgeon nevertheless has considerable leeway in choosing to employ one particular vein and one particular artery in combination, as is shown by FIGS. 16 and 17 respectively.

For these reasons, merely to illustrate the most typical and frequently used combinations of veins and arteries is the non-exhaustive and representative preferred listing of Table 1 below.

TABLE 1
Desirable Combinations

| Choice of vein | Choice of artery |
|---|---|
| Jugular vein | Brachial artery |
| Axillary vein | Axillary artery |
| Femoral vein | Femoral artery |
| Subclavian vein | Subclavian artery |

### IV. The Surgical Method Comprising the Present Invention

### A. An Overview of the Methodology

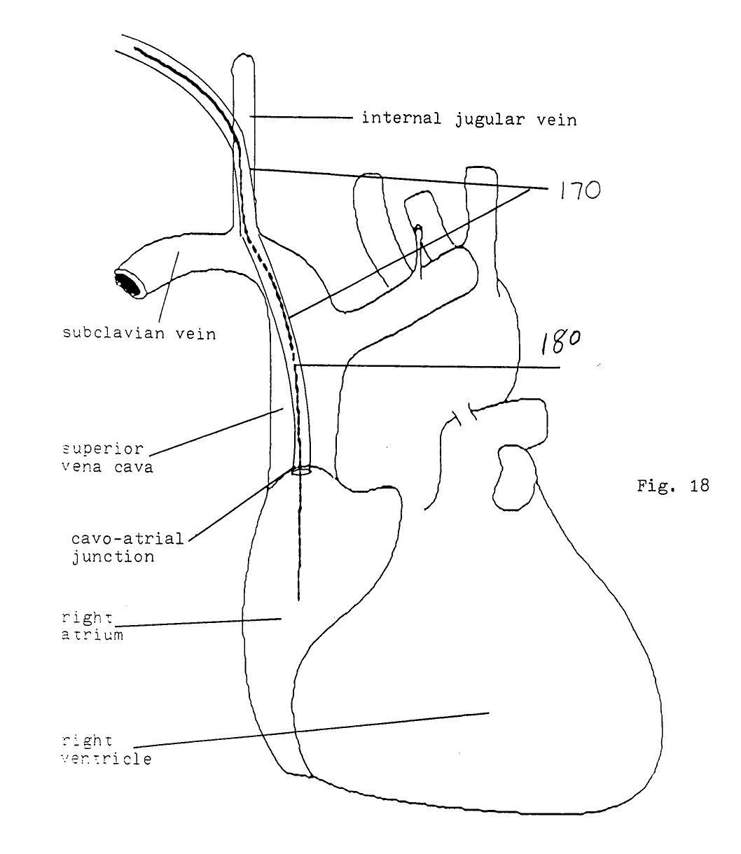

A summary description of the most preferred surgical insertion method—which will be recited again in greater detail hereinafter and is illustrated by FIGS. 18-22 respectively—is the following: A prosthetic endograft is inserted percutaneously (using the vascular balloon catheter as an obturator) into the right jugular vein, and then is passed under fluoroscopic guidance to the level of the cavo-atrial junction of the right atrium. The prosthetic endograft is then subcutaneously tunneled into the arm of the patient from its insertion sue in the right lower neck area; is passed down over the shoulder; and then exits over and into a segment of the right brachial artery for anastamosis. This anastamosis site can vary in anatomic location from just above the elbow crease in the medial bicipital groove, to just below the right axilla, in the proximal bicipital groove. At the selected inflow site, a small incision is made in the skin, the brachial artery is isolated and the proximal anastamosis of the inflow limb of the graft is completed using standard vascular surgical techniques.

The described surgical methodology and insertion technique therefore provides not less than four major benefits and unique advantages:

1. The methodology uses an endovascular approach to create a suture-less venous connection between the endograft and the venous circulation. Thus, a rapid, hemostatic, maximally patent connection is created with this technique. In this minimally invasive way, and by avoiding the standard open surgical techniques, an improved durable connection is made which markedly reduces the risks of potential infection and healing difficulties resulting from a standard conventional surgical procedure.

2. Neointimal hyperplasia, as shown in the radiograph, occurs at the distal anastamosis outflow end of the endograft. By employing a major modification of the “elephant trunk” technique—and because there is no vascular anastamosis between the graft and the outflow venous vessel—the negative pathologic flow dynamics (leading to vascular neointimal hyperplasia, subsequent graft thrombosis, and failure) will be obviated completely. As a consequence, the subsequent long-term patency of these endografts will be significantly greater, and markedly prolong the effective durability and safety of vascular access procedures.

3. In addition, because the venous end (the distal conduit arm) of the endograft is anatomically positioned at the level of the right atrium, potentially higher blood flow rates will be obtained which are not limited by smaller sized veins. This markedly reduces the actual dialysis time for the patient and improves the efficiency of the dialysis process itself.

4. Finally, by utilizing the open and free-floating “elephant trunk” mode of venous connection, if and only if thrombosis of the endograft does occur for other reasons than neointimal hyperplasia, subsequent de-clotting (or thrombectomy) will be more easily facilitated and completed because of the flow dynamics of such a vascular anastamosis.

### B. A Detailed Recitation of the Surgical Insertion Method

For purposes of providing the user with a clear comprehension and better appreciation of the present invention as a whole, a detailed anatomic description of a preferred surgical method and technique for the insertion of a prosthetic endograft is stated below.

It will be expressly understood, however, that the details of the surgical technique described herein, as well as the choices of anatomic location and of specific vein and artery employed, are no more than a preferred embodiment and single example of the method; and as such, are presented solely as one desirable set of representative and illustrative choices for the surgical methodology as a whole. For these reasons, the intended user of the present invention will recognize and acknowledge that a wide range of alternative anatomic locations for insertion is available to the surgeon; and that a substantial variety and range of choice for a particular vein and artery to be used in combination exist (as shown by the listing of Table 1).

### (i) Anatomic Considerations

A general anatomic positioning of the heart and the venous circulation is shown by FIG. 17. The user is presumed to be both cognizant and familiar with the different anatomic locations and positional relationships among the different major veins in the human blood circulatory system and the heart itself. FIG. 17 is therefore merely a convenient guide and reference model embodying conventional human anatomy and medical knowledge.

(ii) The Venous Implantation Component of the Surgical Procedure:

1. Using the conventionally known Seldinger technique (illustrated herein by FIGS. 15A-15F) at a first incision site 300, a needle puncture of the right internal jugular vein is performed, utilizing either a standard anterior or posterior supraclavicular approach. A 0.038 inch flexible guide wire 180 is then passed through the puncture needle 160 and threaded under fluoroscopic control through the cavo-atrial junction and into the right atrium of the patient's heart. This is illustrated in part by FIG. 18.

2. Removing the puncture needle 160 while securing the guide wire 180 in place, a dilator-introducer sheath 170 is then passed over the guide wire, 180 to the level of the cavo-atrial junction of the patient's heart. This step is illustrated by FIG. 18.

3. Using the radiopaque nature of the dilator-sheath 170, the linear distance from the jugular vein entry site to the cavo-atrial junction of the patient's heart can be measured and confirmed using a limited contrast medium injection. This empirically measured linear distance made using radiopaque contrast injection serves as the subject-customized distal conduit length parameter.

4. A pre-sterilized prosthetic endograft 10 is at hand. The preferred prosthesis is comprised of expanded polytetrafluoroethylene; is about fifty five cm in overall linear length; and is about six mm in outer diameter. The pre-formed endograft structurally provides a ribbed medial section 20, a distal conduit arm 30, and a proximal conduit arm 40; and a series of radiographic markers have been disposed over the linear length of the distal conduit arm.

5. The surgeon then carefully measures and cuts the endovascular distal conduit arm 30 of the prosthetic endograft 10 such that its (blood outflow) distal conduit end 32 extends and has the same linear distance (from the junction of the ribbed medial portion 20 over the distal conduit arm) as the empirically measured linear distance made using radiopaque markings. This will provide a patient-customized distal conduit arm length for the endograft, whose distal conduit end, after insertion, will lie properly in anatomic position adjacent to (but not actually within) the cavo-atrial junction of the patient's heart.

5. After the distal conduit arm 30 of the endograft has been properly patent-customized in length, it is inserted over a vascular balloon catheter (the preferred endograft obturator shown by FIGS. 4-7) having at least one internal lumen and being of an appropriate linear length. The patient-customized distal conduit arm 30 is physically inserted, internally extended, and mounted over the balloon lying at the end of the vascular catheter—such that the distal conduit arm 30 (having radiographic markers placed thereon) lies directly over, under and around the linear length of the deflated balloon. The remainder of the endograft structure (the ribbed medial section and the proximal conduit arm) visibly extends from the interior lumen of the vascular balloon catheter into the ambient environment (as shown by FIGS. 8-10).

6. The balloon of the vascular catheter (obturator) is then inflated at will by the surgeon or physician such that it makes physical contact with and forms a tight seal with the circular wall of the distal conduit arm 30 in the endograft. Preferably, the inflated balloon tip will extend 0.5-1.0 cm beyond the end of the distal conduit arm; and will form a tight fit and secure seal with the distal conduit arm of the endograft, so that it will not become dislodged or disengaged when the endograft-catheter coupling is introduced as a discrete assembly and combined unit into the venous system of the patient.

7. With the previously placed guide wire in position in the venous access site, several hollow, hard plastic dilators are now sequentially passed over the guide wire through the percutaneous puncture site in the neck in order to enlarge the skin entry opening—to the degree that the endograft-catheter obturator coupled assembly can then pass over the guidewire into the venous system. Preferably, these dilators are a set of three gradually enlarging hollow, plastic tubes which are individually passed over the guide wire and through the percutaneous skin entry site, thereby progressively enlarging the neck entry site opening.

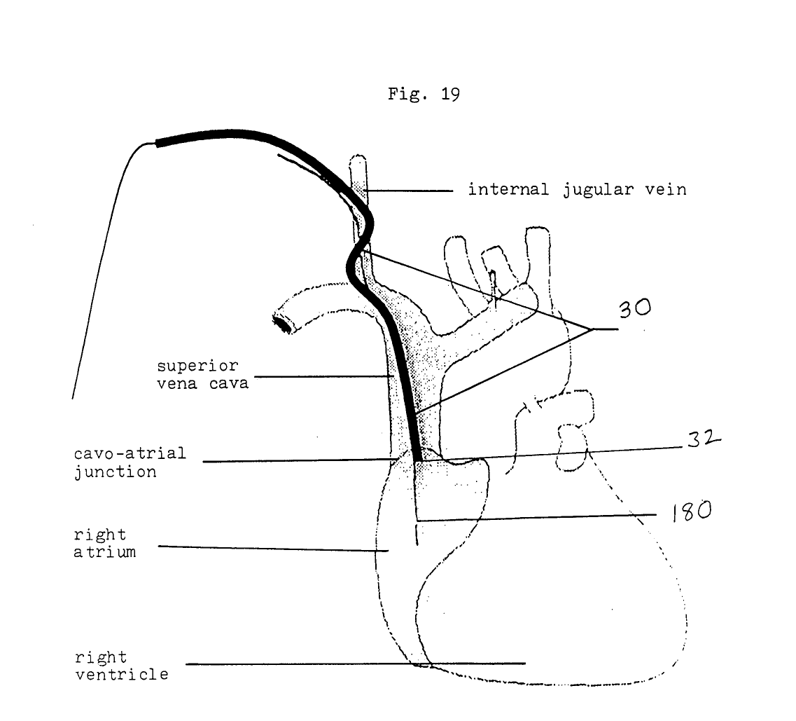

8. After the percutaneous puncture site in the neck has been enlarged to the proper degree, the endograft-balloon catheter coupled assembly and combined unit is passed through the dilated skin entry site into the internal jugular vein, and then extended farther into the superior vena cava of the venous system. Then, the patient-customized distal conduit arm 30 is placed in proper anatomic position at the caval-atrial junction, as seen in FIG. 19. This anatomic positioning is confirmed radiographically using conventionally known methods.

9. Once the endograft-balloon catheter unit is radiographically placed and the distal conduit arm 30 of the endograft lies in fact at the caval-atrial junction, the guide wire is removed from the lumen of the vascular balloon catheter (obturator). The proximal access port of the vascular catheter (through which the wire was removed) can then be used for instillation of one or more contrast agents to confirm proper anatomic position radiographically of the endograft distal end tip.

10. Assuring good anatomic position for the endograft distal end tip, the balloon of the vascular catheter can now be deflated at will; and via this act, the vascular balloon catheter (obturator) becomes separated from, retractable, and completely removable from the endograft structure resting in-situ within the channel of the vein. The acts of separation, retraction, and removal of the vascular balloon catheter from the superior vena cava and the venous system thus result in the stranding and isolation of the distal conduit arm of the endograft within the superior vena cava as the conduit end tip floats freely at the caval-atrial junction of the heart.

11. When this last maneuver (vascular balloon catheter separation, retraction and removal) is performed, blood (from the heart) will flow in retrograde fashion from the right atrium, up into and through the interior of the ribbed medial section and out the proximal conduit arm end of the endograft into the ambient environment. The occurrence of such retrograde blood flow additionally confirms that the intravascular placement of the distal conduit arm of the endograft is anatomically correct and proper. The proximal conduit arm end of the endograft (then visible and exposed to the ambient environment) can now be occluded either by digital pressure or by using a standard vascular bulldog clip to prevent a meaningful loss of blood through the proximal end of the endograft.

Presuming that the physical placement and anatomic location of the distal conduit arm 30 is correct, the custom-sized distal conduit end 32 is now freely floating (without any distal anastomosis as such) at the cavo-atrial junction of the patient's heart. Once proper positioning is confirmed, the venous implantation portion of the surgical methodology is effectively complete.

### (iii) The Tunneling and Subcutaneous Endograft Placement Component of the Surgical Procedure

12. After the proximal conduit arm of the endograft has been clamped to prevent bleeding and/or air from entering into the system, a subcutaneous tunnel will be made in the arm of the patient so that the ribbed medial section 20 and the proximal conduit arm 40 of the endograft can be placed in an in-line position subcutaneously down to the antecubital area of the arm. For achieving this purpose, a small skin incision, approximately 2 cm in length is made with a scalpel over the brachial artery as it is palpated just above the elbow crease on the inner aspect of the arm. This small incision is carried down only to the subcutaneous layer just below the skin, and ends above the muscle and fascia of the arm.

13. Then, using the preferred obturator described previously above as a tunneling tool, the conically-shaped distal tip end of the tool is introduced into the incision just under the skin over the brachial artery. Staying very superficial in the subcutaneous layer, the axial length of the obturator is passed up the arm, making sure that the formed tunnel pathway stays in the same subcutaneous layer by using the natural curve of the obturator to guide the progress of the tunnel.

As the tunnel passageway reaches the area of the shoulder, the distal conical tip of the obturator is aimed at the percutaneous exit site (from which the endograft appears). Again, this pathway should follow the natural curve of the tunneling device, which fits the desired contours of the arm and is the preferred intended placement for the endograft.

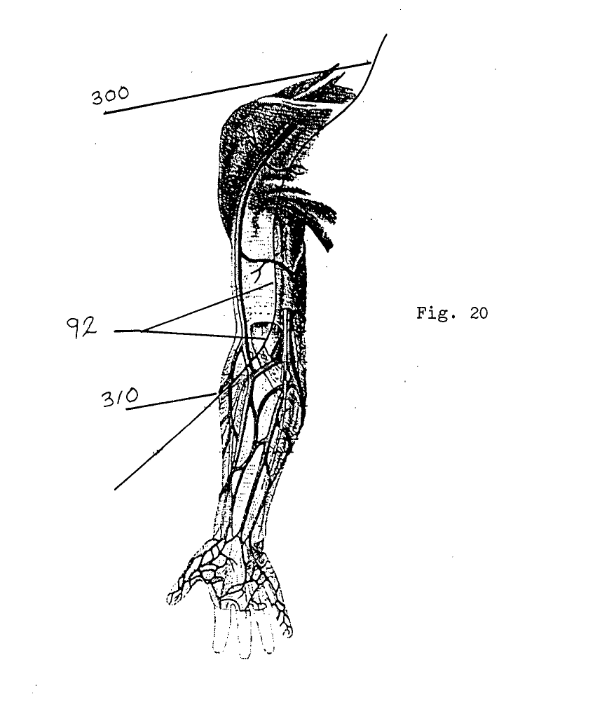

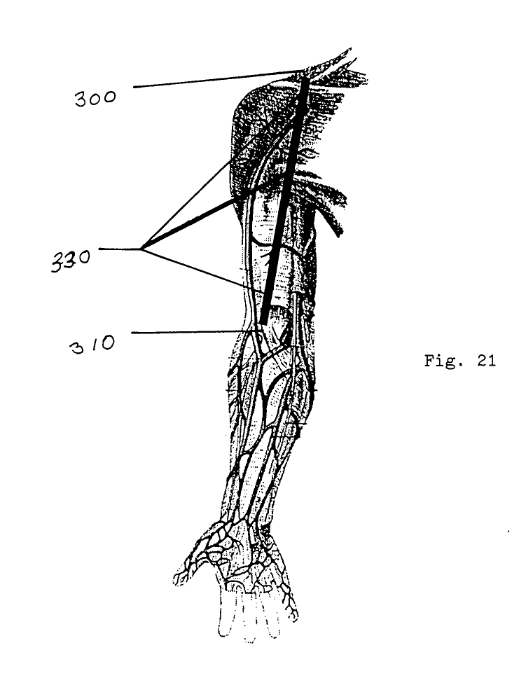

Upon reaching the percutaneous puncture site in the neck (where the endograft exits and emerges), the obturator is maneuvered such that the conically-shaped distal end tip enters the pre-existing puncture site and thereby causes a merger of the newly formed subcutaneous tunnel with the percutaneous puncture site in the neck. This is shown by FIGS. 20-21.

14. After reaching the neck entry incision site (where the endograft is present), the tunneling device is maneuvered to exit from the subcutaneous tunnel within the same neck entry incision site. The end of the proximal conduit arm of the endograft is now threaded over the end of the tunneling device; and is extended onto the distal tip end of the obturator for a distance of about 3 cm, taking care that the extended endograft conduit arm covers the aperture in the conically-shaped tip end of the obturator.

15. At this stage, a suture of any kind of material (but preferably a heavy O-silk suture) is passed through both walls of the extended conduit arm end as well as through the aperture in the conically-shaped tip end of the obturator—thereby effectively skewering the endograft conduit arm to the distal end of the obturator. The same piercing suture is then tied down and circomferentially around the exterior of the endograft conduit arm to complete the primary fixation of the endograft to the obturator.

A secondary suture fixation to reinforce the securing of the endograft is also then preferably performed. A length of suture without a needle is desirably used to then tie and secure the conduit arm of the endograft to the obturator. This second, freely made tie is cinched just above the aperture of the conically-shaped tip; and this second tie further joins and secures the endograft to the distal end of the obturator in order to insure that the endograft will not become inadvertently displaced when the obturator is retracted and withdrawn.

16. After the endograft has been securely fastened to the distal end tip of the tunneling obturator using multiple suture ties, the obturator is then pulled and retracted backwards using hand force through the spatial volume of the newly formed tunnel passageway; and then is completely withdrawn from the small incision site over the brachial artery, an action which concomitantly brings with it the proximal conduit arm and the ribbed medial section of the endograft. Care is taken to be sure the linear length of the endograft does not twist or kink as it travels and that it moves smoothly through the newly created tunnel passageway.

17. In due course, the obturator-endograft connection exits through the incision made distally over the brachial artery. This action also causes the ribbed medial section 20 of the endograft 10 to be pulled into and through the neck (venous) incision site 300.

18. After this occurs, the silk suture ties holding the endograft to the obturator are cut, thereby releasing the endograft entirely from the tunneling obturator. The proximal conduit arm end of the endograft lies exposed and is now ready for anastamosis to the brachial artery.

### (iv) The Arterial Anastomosis Component of the Surgical Procedure

19. Carefully manipulate the ribbed medial portion 20 of the endograft 10 to insure that only a gentle, non-kinked and non-twisted proximal conduit arm lies within the tunnel passageway 330. Also, be sure to allow enough length and linear distance for the proximal conduit arm 40 such that it will lie in a non-stretched manner within the tunnel passageway 330. This is done by moving the proximal conduit arm 40 in abduction and/or adduction so that it does not foreshorten or create any tension within the spatial volume of the tunnel passageway 330.

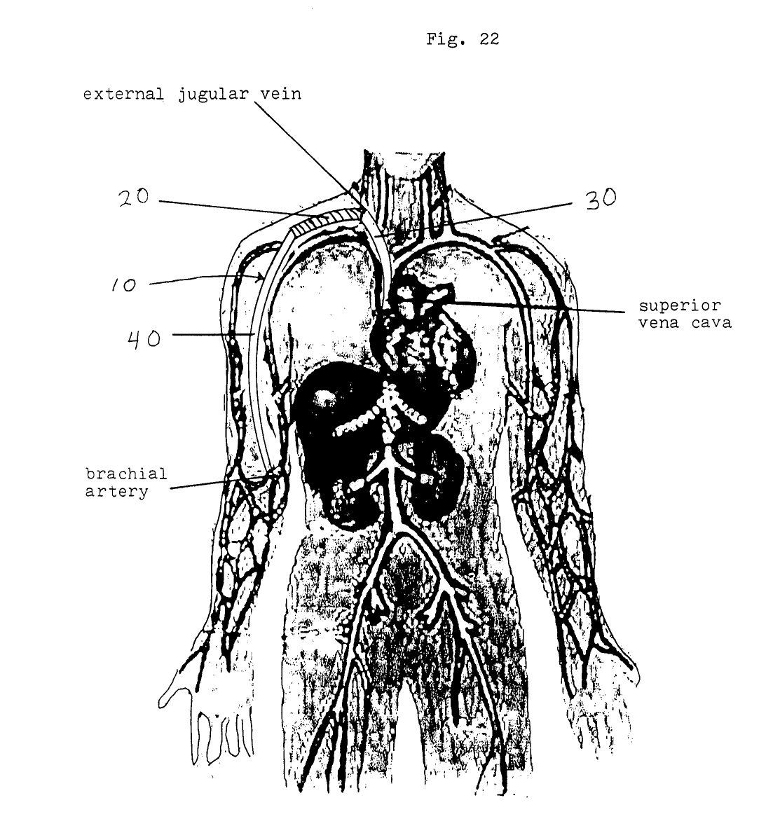

20. Then, carefully measure and custom-cut the proximal conduit arm 40 at the proximal conduit end 44 to the appropriate length such that it will rest directly over the brachial artery. The proximal conduit arm 40 thus is custom-sized in length and is now ready for direct surgical attachment (anastomosis) and fluid flow juncture to the brachial artery. This is shown by FIG. 22.

21. Complete the proximal (inflow) vascular anastamosis to the brachial artery in accordance with conventional surgical technique and medical fashion. Then, de-air the anastamosis; remove the atraumatic graft clamp; and allow blood from the brachial artery to flow through the attached proximal conduit end 44 and proximal conduit arm 40 into the ribbed medial section 20, and then into the distal conduit arm 30 previously positioned at the cavo-atrial junction in the patient's heart.

### (v) Closing of the Skin Incisions and Completion of the Surgical Procedure

22. The two small skin incisions 300, 310 [the venous site incision and the arterial site incision] each are irrigated with a prepared antimicrobial solution; and then are surgically closed in the conventional known and medically appropriate fashion. Standard post-operative follow-up and care is then provided to the patient.

23. The subcutaneously inserted endograft can be used for dialysis access in approximately four weeks time after implantation. The repeated puncture of the ribbed medial section by dialysis needles (for hemodialysis purposes) is self-sealing and markedly limits the risk of hemorrhage.

### V. Critical Requirements of the Surgical Method

### 1. Precise Subject-Customized Sizing of the Distal and Proximal Conduit Arm Linear Lengths in Advance by the Surgeon

The surgeon will size-customize each endograft according to the patient's anatomy and body habitus. The distal end of the prosthetic endograft will be positioned at the atrio-caval junction using the angiographic markers and fluoroscopy. Once that location is determined, the distance from that point to the percutaneous puncture in the neck will be measured. The surgeon will then cut the distal conduit arm of the endograft such that the distance from the beginning of the ribbed portion to the distal conduit end is exactly that measured length. The distal conduit arm of the endograft will then be inserted and positioned into the vein.

The ribbed portion will now begin as the endograft exits the neck incision. The subcutaneous tunnel passageway will exist down the arm and the endograft will be pulled through the tunnel sheath such that it will lie subcutaneously until it exits at the brachial artery incision site. The ribbed portion will be positioned in the area of the neck and shoulder subcutaneously and flexibly so that the endograft does not kink or bend in its course down to the arm. Just above the elbow level where the brachial artery has been identified and dissected free, the endograft will exit the subcutaneous tunnel passageway and be externally visble. Now the surgeon will position, measure and cut the proximal conduit arm such that a properly placed graft-to-artery anastamosis can be performed without kinking or bending and provide an unobstructed blood flow through the endograft.

### 2. Accurate Anatomic Placement of the Distal Conduit End at the Cavo-Atrial Junction

Once the jugular vein has been percutaneously punctured, a guide wire will be inserted through the needle and into the right atrium. A dilator-introducer sheath of radio-opaque material will be threaded over the wire and positioned down the jugular vein and superior vena cava to the level of the atrio-caval junction. Using fluoroscopic guidance and intravascular contrast injections, that site will be accurately identified. Once the tip of the introducer-sheath is positioned at that junction, the distance from the atrio-caval junction to the jugular vein puncture site in the neck will be measured and that distance will now be used to cut the distal endograft to its proper length. The introducer sheath will be removed leaving the guide wire in place and the endograft and angioplasty balloon catheter combined unit will be assembled and then threaded over the wire and positioned at the previously identified and measured atrio-caval junction. The propriety of the anatomic position will again be confirmed with a fluoroscopic contrast injection.

3. The Absence of an Anastomosis at the Distal (Outflow) Conduit End:

After its proper positioning at the atrio-caval junction, the distal end of the endograft will be free floating within the lumen of the superior vena cava. There will be no need of any anastamosis; and normal venous return from the arm, neck and head will occur around the endograft. At the level of the jugular vein where the endograft enters, there will also be no anastamosis. Because of the low pressure venous system, the distensibility of the jugular vein and the fact that the graft entrance site into the jugular vein will be a tight fit because no surgical incision was made, there will be no need for any sutured anastamosis. The venous entry site will seal naturally around the endograft.

4. The Need for an Anastomosis at the Proximal (Inflow) Conduit End:

Once the endograft has been passed through the tunneled passageway subcutaneously through the neck and down the arm, it will exit the tunnel passageway through the surgically made skin incision at the brachial artery site. A point of intended attachment will be chosen on the brachial artery; and the endograft will then be measured and custom-cut so that a standard sutured vascular anastamosis can be performed. This will be an end-to-side vascular anastamosis (end of the endograft sutured to the side of the brachial artery). Once completed, arteriovenous flow will be established from the brachial artery through the endograft interior and into the right atrium.

### VI. Medical Precautions And Potential Complications of the Surgical Method

1. The medical precautions and potential complications will be those of any surgically created A-V vascular access. In general those complications include bleeding at the percutaneous entrance site in the neck, at the surgical incision site, and at the vascular anastamosis in the arm. Additionally, bleeding can occur along and through the subcutaneous tunnel passageway because of potentially disrupted small vessels while creating the tunnel in a blunt manner. Also, thrombosis of the endograft can occur such that flow through the A-V endograft will cease. Furthermore, thrombosis or injury can occur to the native vessels involved, specifically the brachial artery and the jugular vein and/or the superior vena cava.

Infection can occur at any of these sites, the bacteria being introduced at the time of the surgery or at a later date while using the A-V endograft for dialysis. Re-operation may be necessary at various times because of bleeding, thrombosis, anatomic malposition, or kinking of the endograft; and removal may become necessary because of infection or a revision of the graft owing to any or all of the above-mentioned problems.

A “steal” syndrome may also occur in the arm. This is a phenomenon whereby after the A-V endograft has been created and blood flow established, the endograft itself may “steal” blood flow from the distal extremity such that arterial insufficiency is experienced and complications thereof. While uncommon, it can occur and be seen with any surgically created A-V connection.

2. Precautions which can be taken to avoid such complications are also standard; and the same set of precautions that would be performed in any conventionally known surgically created A-V graft for vascular access. These precautions include making sure of the distal endograft placement at the atrio-caval junction using the steps and methods outlined. Additionally, one must make sure that any bleeding or bleeding problems are addressed at the time of operation and properly corrected.

Thrombosis may be avoided by strict attention to prevention of kinking of the endograft in its course from the atrio-caval junction all the way to the brachial artery anastamosis. Identifying and documenting free flow at the end of the procedure using fluoroscopic contrast imaging will also be a preventative step; as well as liberal use of these same methods throughout the procedure to identify proper vessels, locations and configurations.

Infection can be prevented by standard sterile surgical technique as well as the use of pre-operative and post-operative antibiotics in a prophylactic manner. While the “steal” syndrome may not be able to be predicted or prevented, identifying those individuals, like diabetic females, who may be at greater risk for such a complication is useful, so that an awareness of said syndrome is present. Finally, precise and accurate identification, placement, creation and performance of aforementioned steps will be the best preventative measures to avoid complications and problems with this method. As stated previously herein, such potential complications and problems are no different or greater in number than the standard surgical vascular access creation that is performed at present.

### VII. Other Potential Therapeutic Uses and Future Clinical Applications in Addition to Hemodialysis

Clearly hemodialysis is the present and primary focus of the present invention. Nevertheless, there are other clinical applications and therapeutic uses which are envisioned and are deemed to be available at the present time. Additionally, it is expected that there are also a number of future conditions and endeavors which will use this apparatus and methodology to marked advantage.

For these reasons, a listing of present and immediate possible uses for the vascular access provided by the present invention is given by Table 2; and a listing of envisioned clinical applications in the foreseeable future is given by Table 3 below.

TABLE 2
Present and immediate possible uses

Plasmapheresis;
Erythropheresis;
Leucopheresis;
Plateletpheresis;
Long-term instillation of antibiotics;
Chemotherapy treatment; and
Long-term or permanent parenteral hyperalimentation (nutritional
support)

TABLE 3
Envisioned clinical applications in the foreseeable future
Hyperthermic regional chemotherapy;
Monoclonal antibody therapy;
Hepatic hemo-detoxification;
Microsphere-directed radio-tagged, or chemo-tagged, antibody therapy;
Bone marrow transplantation; and
Hypothermic circulatory arrest and/or suspended animation

The present invention is not to be restricted in form nor limited in scope except by the claims appended hereto:

What I claim is:

1. A surgical prosthetic endograft insertion kit whose components are to be used to create a durable vascular access suitable for long-term hemodialysis in a particular subject afflicted with end stage renal disease, said surgical prosthetic endograft insertion kit comprising:
(a) a subject-customized prosthetic endograft suitable for the carrying of flowing blood, which is configured as a flexible, elongated hollow tube and is constructed of at least one durable and biocompatible material, said prosthetic endograft comprising
(i) a hollow ribbed medial section having a predetermined length, external diameter size, tubular wall thickness, and internal lumen diameter, and whose tubular wall can be repeatedly penetrated on-demand by dialysis needles,
(ii) a hollow distal conduit arm having two open ends, one end terminating as a discrete distal conduit end and the other end being integrally joined to and in fluid flow communication with said ribbed medial section, said distal conduit arm being of predetermined external diameter size, tubular wall thickness, and internal lumen diameter, and having a subject-customized linear length which is to be custom-sized by a surgeon such that after in-vivo insertion of said sized distal conduit arm into a pre-chosen vein in the particular subject, said distal conduit end will float freely within the vein and anatomically lie adjacent to the cavo-atrial junction of the heart in the particular subject,
(iii) a hollow proximal conduit arm having two open ends, one end terminating as a discrete proximal conduit end and the other end being integrally joined to and in fluid flow communication with said ribbed medial section, said proximal conduit arm being of predetermined external diameter size, tubular wall thickness, and internal lumen diameter, and having a subject-customized linear length which is to be custom-sized by a surgeon such that said sized proximal conduit arm can be subcutaneously positioned over its entire sized length within the upper limb in a particular subject, and said proximal conduit end can be surgically joined to and anastomosed at a pre-selected anatomic site with a pre-chosen artery in the upper limb of the particular subject;
(b) a vascular balloon catheter formed of durable material and having pre-set dimensions, said vascular balloon catheter comprising a at least one substantially tubular stand having an internal lumen, an access port joined to one end of said tubular strand, and an inflatable and deflatable on-demand balloon disposed at the other end of said tubular strand, wherein said vascular balloon catheter serves as an obturator for said prosthetic endograft and is able to accommodate said distal conduit arm of said endograft over said balloon to form a coupled assembly;
(c) a tunneling obturator system comprising at least one elongated obturator of fixed dimensions and volume having a conically-shaped tip end and which can be employed to form a subcutaneous tunnel passageway within the tissues of the body; and
(d) Seldinger technique workpieces comprising
a Seldinger needle of specific gauge,
a series of graded vein dilators of known linear length and diameter which can be threaded over a guide wire to enlarge the skin and vein entry site; and
a guide wire of specified girth and length.

2. A surgical prosthetic endograft insertion kit whose components are to be used to create a durable vascular access, said surgical prosthetic endograft insertion kit comprising:
(a) a subject-customized prosthetic endograft suitable for the carrying of flowing blood, which is configured as a flexible, elongated hollow tube and is constructed of at least one durable and biocompatible material, said prosthetic endograft comprising
(i) a hollow ribbed medial section having a predetermined length, external diameter size, tubular wall thickness, and internal lumen diameter, and whose tubular wall can be repeatedly penetrated on-demand by syringe needles,
(ii) a hollow distal conduit arm having two open ends, one end terminating as a discrete distal conduit end and the other end being integrally joined to and in fluid flow communication with said ribbed medial section, said distal conduit arm being of predetermined external diameter size, tubular wall thickness, and internal lumen diameter, and having a subject-customized linear length which is to be custom-sized by a surgeon such that after in-vivo insertion of said sized distal conduit arm into a pre-chosen vein in the particular subject, said distal conduit end will float freely within the vein and anatomically lie adjacent to the cavo-atrial junction of the heart in the particular subject,
(iii) a hollow proximal conduit arm having two open ends, one end terminating as a discrete proximal conduit end and the other end being integrally joined to and in fluid flow communication with said ribbed medial section, said proximal conduit arm being of predetermined external diameter size, tubular wall thickness, and internal lumen diameter, and having a subject-customized linear length which is to be custom-sized by a surgeon such that said sized proximal conduit arm can be subcutaneously positioned over its entire sized length within the upper limb in a particular subject, and said proximal conduit end can be surgically joined to and anastomosed at a pre-selected anatomic site with a pre-chosen artery in the upper limb of the particular subject;
(b) a vascular balloon catheter formed of durable material and having pre-set dimensions, said vascular balloon catheter comprising a at least one substantially tubular stand having an internal lumen, an access port joined to one end of said tubular strand, and an inflatable and deflatable on-demand balloon disposed at the other end of said tubular strand, wherein said vascular balloon catheter serves as an obturator for said prosthetic endograft and is able to accommodate said distal conduit arm of said endograft over said balloon to form a coupled assembly;
(c) a tunneling obturator system comprising at least one elongated obturator of fixed dimensions and volume having a conically-shaped tip end and which can be employed to form a subcutaneous tunnel passageway within the tissues of the body; and
(d) Seldinger technique workpieces comprising
a Seldinger needle of specific gauge,
a series of graded vein dilators of known linear length and diameter which can be threaded over a guide wire in order to enlarge the skin and venous entry site, and
a guide wire of specified girth and length.

3. A surgical method for creating a durable vascular access in a living subject suffering from a clinically recognized pathological condition, said surgical method comprising the steps of:
(a) obtaining a subject-customized prosthetic endograft configured as a flexible, elongated hollow tube and constructed of at least one durable and biocompatible material, said prosthetic endograft comprising
(i) a hollow ribbed medial section having a predetermined length, external diameter size, tubular wall thickness, and internal lumen diameter, and whose tubular wall can be repeatedly penetrated on-demand by syringe needles,
(ii) a hollow distal conduit arm having two open ends, one end terminating as a discrete distal conduit end and the other end being integrally joined to and in fluid flow communication with said ribbed medial section, said distal conduit arm being of predetermined external diameter size, tubular wall thickness, and internal lumen diameter, and having a subject-customized linear length which is custom-sized by the surgeon such that after in-vivo insertion of said sized distal conduit arm into a pre-chosen vein in the particular subject, said distal conduit end will float freely within the vein and anatomically lie adjacent to the cavo-atrial junction of the heart in the particular subject,
(iii) a hollow proximal conduit arm having two open ends, one end terminating as a discrete proximal conduit end and the other end being integrally joined to and in fluid flow communication with said ribbed medial section, said proximal conduit arm being of predetermined external diameter size, tubular wall thickness, and internal lumen diameter, and having a subject-customized linear length which is custom-sized by the surgeon such that said sized proximal conduit arm can be subcutaneously positioned over its entire sized length within the upper limb in a particular subject, and said proximal conduit end can be surgically joined to and anastomosed at a pre-selected anatomic site with a pre-chosen artery in the upper limb of the particular subject;
(b) procuring a vascular balloon catheter formed of durable material and having pre-set dimensions, said vascular balloon catheter comprising a at least one substantially tubular stand having an internal lumen, an access port joined to one end of said tubular strand, and an inflatable and deflatable on-demand balloon disposed at the other end of said tubular strand;
(c) passing said prosthetic endograft over said vascular balloon catheter such that said distal conduit arm of said prosthetic endograft is placed over said balloon of said vascular catheter, and then inflating said balloon on-demand to form a coupled assembly;
(d) percutaneously passing said coupled assembly through a first insertion site at a pre-selected anatomic position into the internal channel of the pre-chosen vein in the living subject, whereby said distal conduit arm of said coupled assembly comes to rest entirely within the channel of the pre-chosen Vein, and whereby said distal conduit arm end floats freely and anatomically lies within the pre-chosen vein adjacent to the cavo-atrial junction of the heart in the living subject;
(e) deflating said balloon of said vascular balloon catheter on-demand to release said anatomically positioned distal conduit arm of said prosthetic endograft from said coupled assembly and then removing said vascular balloon catheter from the vein without displacing said anatomically positioned distal conduit arm;
(f) creating a second insertion site at a second pre-selected anatomic position in the upper limb of the particular subject to gain access to a pre-chosen artery in the upper limb of the particular subject;
(g) surgically forming a subcutaneous tunnel passageway within the upper limb which extends upwardly from said second insertion site and terminates adjacent to the first insertion site in the neck/shoulder of the particular patient, said formed subcutaneous tunnel and open passageway being substantially parallel to the anatomic location of the pre-chosen artery within the upper limb;
(h) passing said proximal conduit arm of said prosthetic endograft into and through the length of said subcutaneous tunnel and open passageway such that said custom-sized proximal conduit end lies adjacent to said second insertion site on the upper limb of the particular patient;
(i) introducing said ribbed medial section of said prosthetic endograft through said first insertion site such said ribbed medial section lies subcutaneously adjacent to said open passageway and subcutaneous tunnel; and
(j) joining said custom-sized proximal conduit end to said pre-chosen artery in the upper limb of the particular subject.

4. The method as recited in claim 3 wherein the clinically recognized condition is one selected from the group consisting of plasmapheresis, erythropheresis, leucopheresis, platletpheresis, long-term instillation of antibiotics, chemotherapy treatment, and parenteral hyperalimentation.

5. The method as recited in claim 3 wherein the clinically recognized condition is one selected from the group consisting of hyperthermic region chemotherapy, monoclonal antibody therapy, hepatic hemo-detoxification, micro-sphere-directed antibody therapy, bone marrow transplantation, hypothermic circulatory arrest, and suspended animation.

6. A surgical method for creating a durable vascular access suitable for long-term hemodialysis in a living subject afflicted with end stage renal disease, said surgical method comprising the steps of:
(1) creating a first insertion site at a pre-selected anatomic position in the neck/shoulder of the living subject to percutaneously puncture a pre-chosen vein;
(2) preparing a subject-customized prosthetic endograft configured as a flexible, elongated hollow tube and constructed of at least one durable and biocompatible material, said prosthetic endograft comprising
(i) a hollow ribbed medial section having a predetermined length, external diameter size, tubular wall thickness, and internal lumen diameter, and whose tubular wall can be repeatedly penetrated on-demand by dialysis needles,
(ii) a hollow distal conduit arm having two open ends, one end terminating as a discrete distal conduit end and the other end being integrally joined to and in fluid flow communication with said ribbed medial section, said distal conduit arm being of predetermined external diameter size, tubular wall thickness, and internal lumen diameter, and having a subject-customized linear length which is custom-sized by the surgeon such that after in-vivo insertion of said sized distal conduit arm into a pre-chosen vein in the particular subject, said distal conduit end will float freely within the vein and anatomically lie adjacent to the cavo-atrial junction of the heart in the particular subject,
(iii) a hollow proximal conduit arm having two open ends, one end terminating as a discrete proximal conduit end and the other end being integrally joined to and in fluid flow communication with said ribbed medial section, said proximal conduit arm being of predetermined external diameter size, tubular wall thickness, and internal lumen diameter, and having a subject-customized linear length which is custom-sized by the surgeon such that said sized proximal conduit arm can be subcutaneously positioned over its entire sized length within the upper limb in a particular subject, and said proximal conduit end can be surgically joined to and anastomosed at a pre-selected anatomic site with a pre-chosen artery in the upper limb of the particular subject;
(3) procuring a vascular balloon catheter formed of durable material and having pre-set dimensions, said vascular balloon catheter comprising a at least one substantially tubular stand having an internal lumen, an access port joined to one end of said tubular strand, and an inflatable and deflatable on-demand balloon disposed at the other end of said tubular strand;
(41) passing said prosthetic endograft over said vascular balloon catheter such that said distal conduit arm of said prosthetic endograft is placed over said balloon of said vascular catheter, and then inflating said balloon on-demand to form a coupled assembly;
(5) percutaneously passing said coupled assembly through a first insertion site at a pre-selected anatomic position into the internal channel of the pre-chosen vein in the living subject, whereby said distal conduit arm of said coupled assembly comes to rest entirely within the channel of the pre-chosen Vein, and whereby said distal conduit arm end floats freely within and anatomically lies within the vein adjacent to the cavo-atrial junction of the heart in the living subject;
(6) deflating said balloon of said vascular balloon catheter on-demand to release said anatomically positioned distal conduit arm of said prosthetic endograft from said coupled assembly and then removing said vascular balloon catheter from the vein without displacing said anatomically positioned distal conduit arm;
(7) creating a second insertion site at a second pre-selected anatomic position in the upper limb of the particular subject to gain access to a pre-chosen artery in the upper limb of the particular subject;
(8) mobilizing a segment of the accessed pre-chosen artery in the upper limb of the particular subject;
(9) surgically forming a subcutaneous tunnel passageway within the upper limb which extends upwardly from said second insertion site and terminates adjacent to the first insertion site in the neck/shoulder of the particular patient, said formed subcutaneous tunnel and open passageway being substantially parallel to the anatomic location of the pre-chosen artery within the upper limb;
(10) passing said proximal conduit arm of said prosthetic endograft into and through the length of said subcutaneous tunnel and open passageway such that said custom-sized proximal conduit end lies adjacent to said second insertion site on the upper limb of the particular patient;
(11) introducing said ribbed medial section of said prosthetic endograft through said first insertion site such said ribbed medial section lies subcutaneously adjacent to said open passageway and subcutaneous tunnel; and
(12) joining and anastomosing said custom-sized proximal conduit end to said mobilized segment of the pre-chosen artery in the upper limb of the particular subject; and
(13) surgically closing said first and second insertion sites.
